(19) United States
(12) Patent Application Publication
(10) Pub. No.: US 2026/0248429 A1
(43) Pub. Date: Aug. 27, 2026

(54) SYSTEM

(71) Applicant: SoftBank Group Corp., Tokyo (JP)

(72) Inventor: Shiho HAMAGUCHI, Tokyo (JP)

(73) Assignee: SoftBank Group Corp., Tokyo (JP)

(21) Appl. No.: 19/534,804

(22) Filed: Feb. 10, 2026

(30) Foreign Application Priority Data
Feb. 21, 2025 (JP) 2025-027079

(51) Int. Cl. A61B 5/16; A61B 5/00; A61M 21/02; G06V 10/82; G06V 40/16; G10L 25/30; G10L 25/63; G16H 80/00

(52) U.S. Cl. CPC A61B 5/165; A61B 5/4803; A61B 5/4836; A61B 5/7267; A61M 21/02; A61M 2205/3303; G06V 10/82; G06V 40/174; G10L 25/30; G10L 25/63; G16H 80/00

(57) ABSTRACT

The system according to the embodiment comprises a collection unit, an analysis unit, a generation unit, and a provision unit. The collection unit collects health data. The analysis unit analyzes data collected by the collection unit. The generation unit generates advice based on an analysis result obtained by the analysis unit. The provision unit provides relaxation or mental health guidance based on the advice generated by the generation unit.

### CROSS-REFERENCE TO RELATED APPLICATION

The present application claims priority to and incorporates by reference the entire contents of Japanese Patent Application No. 2025-027079 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, real-time tracking of individual health data and provision of appropriate advice have not been sufficiently performed, leaving room for improvement.

### SUMMARY OF THE INVENTION

The system according to the embodiment comprises a collection unit, an analysis unit, a generation unit, and a provision unit. The collection unit collects health data. The analysis unit analyzes data collected by the collection unit. The generation unit generates advice based on an analysis result obtained by the analysis unit. The provision unit provides relaxation or mental health guidance based on the advice generated by the generation unit.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

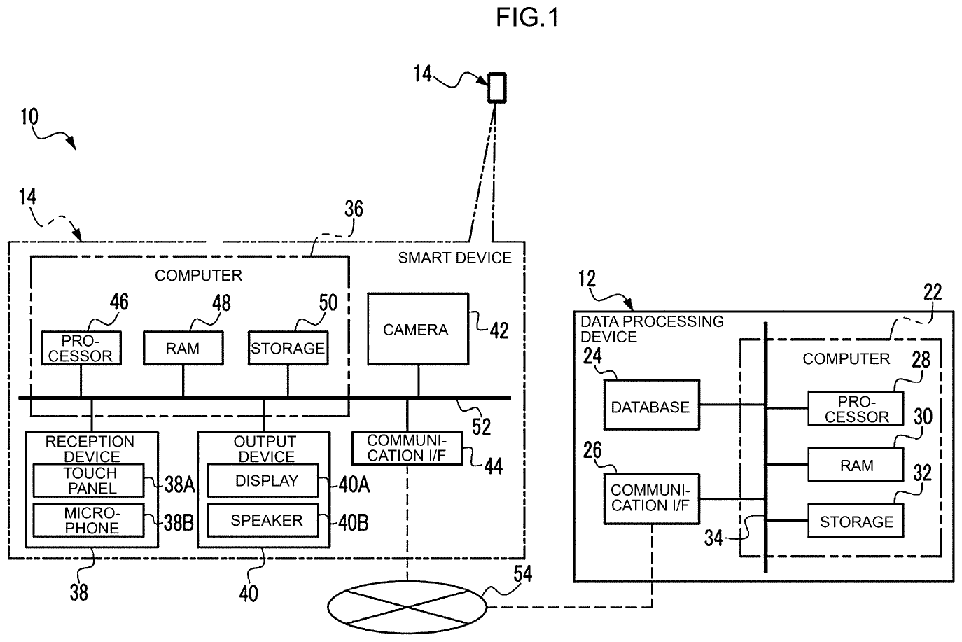

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

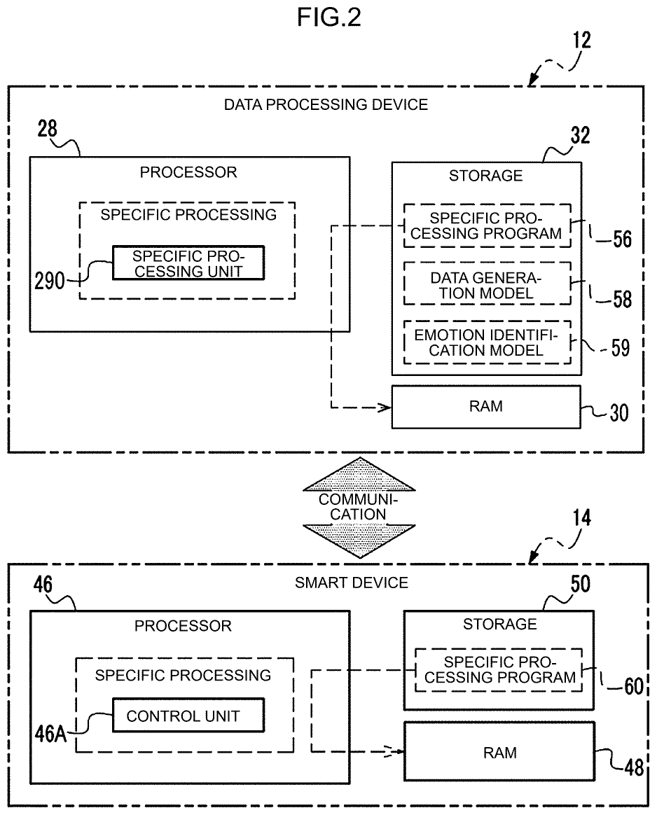

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example of the Embodiment

The health management support system according to the embodiment of the present invention is a system that tracks a user's health data in real time and provides appropriate exercise and nutrition advice. This system also provides relaxation and mental health guidance to improve physical and mental balance by monitoring heart rate and stress level. For example, the user wears a wearable device. This device is equipped with a heart rate sensor and a sensor configured to measure a stress level, and these sensors collect the user's health data in real time. For instance, the heart rate sensor constantly monitors the user's heart rate and issues an alert when abnormal values are detected. Next, the collected health data is analyzed by AI within the device. The AI evaluates the user's health condition and generates appropriate exercise and nutrition advice. For example, if the user's heart rate is high, advice for deep breathing to relax is provided. Additionally, the AI analyzes the user's dietary data and, if the nutritional balance is biased, proposes a balanced diet. Furthermore, the AI monitors the user's stress level and provides relaxation or mental health guidance as needed. For example, if the stress level is high, guidance for meditation or yoga is provided to support improvement of physical and mental balance. In this way, the wearable device equipped with AI tracks the user's health data in real time and provides appropriate advice to support individual health management. As a result, the health management support system can track the user's health data in real time, provide appropriate exercise and nutrition advice, and offer relaxation and mental health guidance to improve physical and mental balance. Specifically, the health management support system inputs multidimensional biosignal data (e.g., time-series heart rate data per second, continuous skin electrical activity values, triaxial acceleration data, etc.) obtained from multiple sensors (e.g., optical heart rate sensor, skin electrical activity sensor, accelerometer, etc.) mounted on the wearable device into an edge AI processor within the device or a large language model on the cloud. The system first performs preprocessing such as noise removal and normalization (e.g., heart rate noise removal by bandpass filter, Z-score normalization, etc.) on the input data, and then extracts features using convolutional neural networks (CNN), recurrent neural networks (RNN), or Transformer-based time-series analysis models. Examples of AI inputs include (1) a 30-second heart rate time-series vector (e.g., a real-valued array of 30 elements), (2) one minute of sampled skin electrical activity data (e.g., 60 continuous values), and (3) text data of meal records (e.g., “Breakfast: bread, eggs, milk”). The AI generates outputs such as (a) health score (e.g., a value from 0 to 100), (b) anomaly detection label (e.g., “Heart rate anomaly: Yes”), and (c) recommended actions (e.g., “Recommend deep breathing,” “Recommend balanced diet”). Example outputs include “Health score: 82,” “No anomaly detected,” “Recommended exercise: 20 minutes walking,” “Recommended diet: increase vegetables.” These outputs are passed to a threshold judgment module or user interface module, and if the threshold is exceeded, an alert is issued, and recommended actions are notified to the user's smartphone or wearable device screen. Internally, the AI model uses loss functions such as mean squared error or cross-entropy loss and updates weights by gradient descent to continuously improve the accuracy of health condition estimation and anomaly detection. Furthermore, for individual optimization for each user, transfer learning and online learning methods are applied, and the user's past data and feedback are sequentially reflected in learning. In conventional human health management, data collection, analysis, and advice generation were fragmented and subjective, but this system enables integrated analysis of high-dimensional data, nonlinear pattern recognition, and real-time processing, thereby providing the technical effect of instantly detecting changes in health condition and automatically generating individually optimized advice. Application fields include health management for general consumers, employee health support for companies, condition management for athletes, and rehabilitation support in medical institutions. Thus, the present invention achieves not only automation of human tasks but also essential improvement of computer technology for high-precision and high-efficiency health management support by AI.

The health management support system according to the embodiment comprises a collection unit, an analysis unit, a generation unit, and a provision unit. The collection unit collects health data of a user. The user's health data may include, for example, heart rate, blood pressure, and stress level, but is not limited to these examples. The collection unit collects the user's health data in real time using, for example, a heart rate sensor or a sensor configured to measure a stress level. The heart rate sensor measures heart rate using, for example, an optical sensor or an electrical sensor. The sensor configured to measure a stress level measures stress level using, for example, a skin electrical activity sensor or a heart rate variability sensor. The analysis unit analyzes health data collected by the collection unit. The analysis is performed using, for example, statistical analysis or machine learning algorithms. The analysis unit analyzes the collected health data and evaluates the health condition of the user. The evaluation of health condition includes, for example, calculation of a health score and detection of abnormal values. The generation unit generates advice based on an analysis result obtained by the analysis unit. The advice includes, for example, exercise proposals and dietary recommendations. The generation unit generates appropriate exercise or nutrition advice based on the health condition of the user. The provision unit provides relaxation or mental health guidance based on the advice generated by the generation unit. Relaxation or mental health guidance includes, for example, meditation instruction and stress management techniques. The provision unit provides relaxation or mental health guidance based on the stress level of the user. Thus, the health management support system according to the embodiment can track the user's health data in real time, provide appropriate exercise and nutrition advice, and offer relaxation or mental health guidance to improve physical and mental balance. Specifically, the health management support system inputs multidimensional biosignal data (e.g., time-series heart rate data per second, continuous skin electrical activity values, triaxial acceleration data, continuous blood pressure measurements, etc.) obtained from multiple sensors (e.g., optical heart rate sensor, skin electrical activity sensor, accelerometer, blood pressure sensor, etc.) mounted on wearable devices or smartphones into an edge AI processor within the device or a large language model on the cloud. The system first performs preprocessing such as noise removal and normalization (e.g., heart rate noise removal by bandpass filter, Z-score normalization, outlier removal, etc.) on the input data, and then extracts features using convolutional neural networks (CNN), recurrent neural networks (RNN), or Transformer-based time-series analysis models. Examples of AI inputs include (1) a 30-second heart rate time-series vector (e.g., a real-valued array of 30 elements), (2) one minute of sampled skin electrical activity data (e.g., 60 continuous values), (3) text data of meal records (e.g., “Breakfast: bread, eggs, milk”), and (4) continuous blood pressure measurements (e.g., a 10-minute array of blood pressure values). The analysis unit generates outputs such as (a) health score (e.g., a value from 0 to 100), (b) anomaly detection label (e.g., “Heart rate anomaly: Yes”), and (c) recommended actions (e.g., “Recommend deep breathing,” “Recommend balanced diet”). Example outputs include “Health score: 82,” “No anomaly detected,” “Recommended exercise: 20 minutes walking,” “Recommended diet: increase vegetables.” These outputs are passed to a threshold judgment module or user interface module, and if the threshold is exceeded, an alert is issued, and recommended actions are notified to the user's smartphone or wearable device screen. Internally, the AI model uses loss functions such as mean squared error or cross-entropy loss and updates weights by gradient descent to continuously improve the accuracy of health condition estimation and anomaly detection. Furthermore, for individual optimization for each user, transfer learning and online learning methods are applied, and the user's past data and feedback are sequentially reflected in learning. In conventional human health management, data collection, analysis, and advice generation were fragmented and subjective, but this system enables integrated analysis of high-dimensional data, nonlinear pattern recognition, and real-time processing, thereby providing the technical effect of instantly detecting changes in health condition and automatically generating individually optimized advice. Application fields include health management for general consumers, employee health support for companies, condition management for athletes, and rehabilitation support in medical institutions. Thus, the present invention achieves not only automation of human tasks but also essential improvement of computer technology for high-precision and high-efficiency health management support by AI.

The collection unit may comprise a heart rate sensor or a sensor configured to measure a stress level. The collection unit may measure heart rate using, for example, a heart rate sensor. The heart rate sensor includes optical sensors and electrical sensors. The optical sensor detects blood flow using light and measures heart rate. The electrical sensor measures heart rate using electrical signals. The collection unit may also measure stress level using a sensor configured to measure a stress level. Such sensors include skin electrical activity sensors and heart rate variability sensors. The skin electrical activity sensor detects electrical changes on the skin surface and measures stress level. The heart rate variability sensor analyzes heart rate variability and measures stress level. By measuring heart rate and stress level, the collection unit can collect the user's health data more accurately. Some or all of the above-described processing in the collection unit may be performed using AI or without using AI. For example, the collection unit may input data obtained from the heart rate sensor or the sensor configured to measure a stress level into a generative AI and have the generative AI perform data analysis. Specifically, the collection unit preprocesses time-series data of reflected light intensity obtained from an optical heart rate sensor (e.g., a real-valued array of 30 elements sampled every second) and electrocardiogram waveform data obtained from an electrical heart rate sensor (e.g., time-series data of 2,500 elements sampled at 500 Hz) using noise removal and peak detection algorithms (e.g., moving average filter, low-pass filter, R-wave detection algorithm, etc.). From the skin electrical activity sensor, continuous values of skin conductance (e.g., a vector of 60 samples per minute) are obtained, and from the heart rate variability sensor, time-series data of RR intervals (e.g., a real-valued array of 30 elements) are obtained. These multidimensional biosignal data are input into an edge AI processor within the collection unit or a large language model on the cloud. Examples of AI inputs include (1) a 30-second heart rate time-series vector, (2) one minute of continuous skin electrical activity values, (3) electrocardiogram waveform data, and (4) RR interval data. The AI generates outputs such as (a) anomaly detection label for heart rate (e.g., “Heart rate anomaly: Yes”), (b) estimated stress level (e.g., score from 0 to 100), and (c) biosignal quality evaluation (e.g., “High noise,” “Normal”). Example outputs include “Heart rate: 85 bpm,” “Stress level: 72,” “Signal quality: Normal.” These outputs are passed to the subsequent analysis unit or alert issuing module, and if the threshold is exceeded, the user is notified in real time. Internally, the AI model extracts time-series features using convolutional neural networks (CNN) and analyzes long-term dependencies using recurrent neural networks (RNN), and learns using loss functions such as mean squared error or cross-entropy loss. Furthermore, the collection unit applies online learning or transfer learning for individual optimization for each user, and sequentially reflects the user's past data and feedback. High-precision anomaly detection and stress estimation, which were difficult with conventional manual measurement or simple threshold judgment by humans, can be realized by multidimensional data analysis and real-time processing by AI, resulting in significant improvement in the accuracy and responsiveness of health management. Application fields include health management for general consumers, condition management for athletes, monitoring in medical institutions, and employee health support for companies.

The analysis unit can analyze the collected health data and evaluate the health condition of the user. The analysis unit may perform statistical analysis of the collected health data. Statistical analysis includes calculation of mean and standard deviation of the data. The analysis unit may also analyze health data using machine learning algorithms. Machine learning algorithms include, for example, regression analysis and clustering. Regression analysis is a method for modeling relationships in data and making predictions. Clustering is a method for grouping data. The analysis unit analyzes the collected health data and evaluates the health condition of the user. Evaluation of health condition includes, for example, calculation of a health score and detection of abnormal values. The health score quantifies the user's health condition, and abnormal value detection identifies abnormal values in the health data. By analyzing the collected health data, the health condition of the user can be evaluated. Some or all of the above-described processing in the analysis unit may be performed using AI or without using AI. For example, the analysis unit may input the collected health data into a generative AI and have the generative AI perform health condition evaluation. Specifically, the analysis unit preprocesses multidimensional biosignal data received from the collection unit (e.g., heart rate time-series vector, continuous skin electrical activity values, continuous blood pressure measurements, meal record text, etc.) using noise removal and normalization (e.g., bandpass filter, Z-score normalization, outlier removal). Next, features are extracted using convolutional neural networks (CNN), recurrent neural networks (RNN), or Transformer-based time-series analysis models. Examples of AI inputs include (1) a 30-second heart rate time-series vector (30 real-valued elements), (2) one minute of continuous skin electrical activity values (60 elements), (3) text data of meal records (“Lunch: salad, chicken, rice”), and (4) continuous blood pressure measurements (10-minute array). The analysis unit generates outputs such as (a) health score (0 to 100), (b) anomaly detection label (“Heart rate anomaly: Yes”), and (c) cluster label (“High stress group,” “Low stress group”). Example outputs include “Health score: 78,” “Abnormal value detected: high heart rate,” “Cluster: high stress group.” These outputs are passed to the subsequent generation unit or alert issuing module, and if the health condition exceeds the threshold, an alert is issued. Internally, the AI model uses loss functions such as mean squared error or cross-entropy loss and updates weights by gradient descent to continuously improve the accuracy of health condition estimation and anomaly detection. Furthermore, for individual optimization for each user, transfer learning and online learning methods are applied, and the user's past data and feedback are sequentially reflected in learning. Conventional human health condition evaluation relied on subjective judgment and simple threshold judgment, but the analysis unit enables integrated analysis of high-dimensional data, nonlinear pattern recognition, and real-time processing, thereby providing the technical effect of instantly detecting changes in health condition and automatically generating individually optimized evaluation. Application fields include health management for general consumers, condition management for athletes, rehabilitation support in medical institutions, and employee health support for companies.

The generation unit can generate appropriate exercise or nutrition advice based on the health condition of the user. The generation unit may propose exercise based on the health condition of the user. Exercise proposals include the type, intensity, and frequency of exercise. For example, if the user's heart rate is high, the generation unit provides advice for deep breathing to relax. The generation unit may also generate nutrition advice based on the health condition of the user. Nutrition advice includes the type, amount, and balance of meals. For example, the generation unit analyzes the user's dietary data and, if the nutritional balance is biased, proposes a balanced diet. By generating appropriate exercise and nutrition advice based on the health condition of the user, the generation unit can support the user's health management. Some or all of the above-described processing in the generation unit may be performed using AI or without using AI. For example, the generation unit may input the user's health condition data into a generative AI and have the generative AI generate exercise and nutrition advice. Specifically, the generation unit inputs health scores, anomaly detection labels, meal record text, exercise history data, etc. received from the analysis unit and first evaluates the user's health condition from multiple perspectives. Examples of AI inputs include (1) health score (e.g., 82), (2) anomaly detection label (e.g., “Heart rate anomaly: Yes”), (3) meal record text (e.g., “Lunch: salad, chicken, rice”), and (4) exercise history (e.g., exercise intensity vector for the past week). The generation unit generates outputs such as (a) recommended exercise (e.g., “20 minutes walking,” “Recommend deep breathing”), (b) recommended diet (e.g., “Increase vegetables,” “Add protein”), and (c) nutrition balance evaluation (e.g., “Excess carbohydrates”). Example outputs include “Recommended exercise: 10 minutes stretching,” “Recommended diet: increase fish,” “Nutrition balance: excess fat.” These outputs are passed to the subsequent provision unit or user interface module and notified to the user. Internally, the AI model uses a Transformer-based large language model to extract nutrients from meal record text and generate optimal advice by combining exercise history and health score. Loss functions such as cross-entropy loss or custom reward functions are used, and reinforcement learning or transfer learning is applied to achieve individual optimization reflecting user preferences and past feedback. Conventional human advice generation relied on subjective judgment and empirical rules, but the generation unit enables integrated analysis of high-dimensional data, nonlinear pattern recognition, and real-time processing, thereby providing the technical effect of automatically generating individually optimized advice in response to changes in health condition. Application fields include health management for general consumers, training support for athletes, rehabilitation guidance in medical institutions, and employee health support for companies.

The provision unit can provide relaxation or mental health guidance based on the stress level of the user. For example, if the user's stress level is high, the provision unit provides relaxation guidance. Relaxation guidance includes deep breathing, meditation, yoga, and the like. For example, the provision unit instructs the user on deep breathing methods and supports relaxation. The provision unit may also provide mental health guidance based on the user's stress level. Mental health guidance includes stress management techniques and mental health care methods. For example, the provision unit instructs the user on stress management techniques and supports improvement of physical and mental balance. By providing relaxation or mental health guidance based on the user's stress level, the provision unit can improve physical and mental balance. Some or all of the above-described processing in the provision unit may be performed using AI or without using AI. For example, the provision unit may input the user's stress level data into a generative AI and have the generative AI generate relaxation or mental health guidance. Specifically, the provision unit inputs recommended actions, stress level scores, health scores, etc. received from the generation unit and selects and generates optimal guidance according to the user's stress state. Examples of AI inputs include (1) stress level score (e.g., 85), (2) health score (e.g., 78), (3) past guidance history (e.g., guidance implementation records for the past week), and (4) user preference information (e.g., likes meditation, dislikes yoga). The provision unit generates outputs such as (a) recommended relaxation guidance (e.g., “Deep breathing guide,” “Meditation guide”), (b) recommended mental health guidance (e.g., “Stress management techniques,” “Mindfulness practice”), and (c) guidance implementation timing (e.g., “Recommended before bedtime”). Example outputs include “Recommended guide: deep breathing method,” “Implementation timing: lunch break,” “Guide content: 3-minute breathing technique.” These outputs are notified to the user's smartphone or wearable device screen, voice assistant, etc. Internally, the AI model uses a large language model to integratively analyze the user's stress level, preferences, and past guidance history and generate optimal guidance content and expression methods. Loss functions such as cross-entropy loss or custom reward functions are used, and reinforcement learning or transfer learning is applied to achieve individual optimization reflecting user reactions and feedback. Conventional human guidance provision relied on subjective judgment and empirical rules, but the provision unit enables integrated analysis of high-dimensional data, nonlinear pattern recognition, and real-time processing, thereby providing the technical effect of automatically generating individually optimized guidance in response to changes in stress state. Application fields include mental health support for general consumers, stress care for company employees, rehabilitation support in medical institutions, and mental conditioning for athletes.

The collection unit can estimate the user's emotion and adjust the timing for collecting health data based on the estimated emotion of the user. For example, if the user is feeling stressed, the collection unit collects health data more frequently during periods of high stress level. The collection unit uses emotion estimation functions, such as an emotion engine or generative AI, to estimate the user's emotion. For example, the collection unit estimates the user's emotion using facial recognition technology. Facial recognition technology analyzes facial data captured by a camera to estimate emotion. The collection unit may also estimate the user's emotion using voice analysis technology. Voice analysis technology analyzes the tone and speed of the user's voice to estimate emotion. Furthermore, if the user is relaxed, the collection unit reduces the frequency of health data collection and collects only necessary data. For example, if the user is exercising, the collection unit collects health data at the start and end of exercise to evaluate the effect of exercise. By adjusting the timing for collecting health data based on the user's emotion, the collection unit can collect data at more appropriate times. Some or all of the above-described processing in the collection unit may be performed using AI or without using AI. For example, the collection unit may input the user's emotion data into a generative AI and have the generative AI adjust the timing for collecting health data. Specifically, the collection unit integratively utilizes multiple sensor data for emotion estimation. The collection unit uses facial landmark points obtained from camera images (e.g., 68-point facial feature vector), acoustic features extracted from voice waveforms (e.g., 20-dimensional vector such as MFCC, pitch, speech rate), and biosignals (e.g., heart rate, skin electrical activity) as time-series tensors for input data. The collection unit first preprocesses these multidimensional data by noise removal and normalization (e.g., face image alignment, voice normalization, Z-score normalization of time-series data). For emotion estimation, the collection unit uses convolutional neural networks (CNN), recurrent neural networks (RNN), or Transformer-based multimodal emotion recognition models. Examples of AI inputs include (1) facial expression feature vectors per second (68 dimensions×30 frames), (2) 3 seconds of voice MFCC features (20 dimensions×30 frames), and (3) 30 seconds of heart rate time-series (30 elements). The collection unit generates outputs such as (a) emotion label (e.g., “Stress,” “Relaxation,” “Anger”), (b) emotion score (e.g., continuous value from 0 to 100), and (c) emotion trend (e.g., “Stress increasing”). Example outputs include “Emotion: Stress,” “Emotion score: 85,” “Emotion trend: Increasing.” Based on these outputs, the collection unit passes data to the health data collection timing control module, and dynamically adjusts collection frequency, such as every minute during high stress and every ten minutes during relaxation. The collection unit collaborates with accelerometer data and GPS data during exercise to automatically detect exercise start and end events and intensively collect data such as heart rate and calories burned at those times. Internally, the AI model uses cross-entropy loss or mean squared error to continuously improve emotion estimation accuracy, and applies transfer learning or online learning using user feedback and past emotion history. Unlike conventional subjective emotion observation and fixed data collection schedules by humans, the collection unit achieves optimal data collection responsive to the user's psychological state through high-dimensional multimodal data analysis and real-time control by AI. Application fields include health management for general consumers, stress monitoring, psychological state management for athletes, and mental health support in medical institutions. Thus, the present invention achieves not only automation of human tasks but also essential improvement of computer technology for emotion recognition and data collection optimization by AI.

The collection unit can analyze the user's past health data and select an appropriate collection method. For example, the collection unit analyzes the user's past heart rate data and increases the frequency of heart rate collection if abnormal patterns are observed. The collection unit may use AI to analyze the user's past health data. For example, the collection unit analyzes past data using machine learning algorithms to detect abnormal patterns. The collection unit may also analyze the user's past stress level data and collect data intensively during periods of high stress. For example, the collection unit predicts stress level fluctuations based on past data and adjusts the collection timing. Furthermore, the collection unit analyzes the user's past exercise data and strengthens data collection after exercise to monitor recovery status. For example, the collection unit analyzes changes in heart rate and stress level after exercise to evaluate recovery status. By analyzing the user's past health data, the collection unit can select the optimal collection method. Some or all of the above-described processing in the collection unit may be performed using AI or without using AI. For example, the collection unit may input past health data into a generative AI and have the generative AI select the optimal collection method. Specifically, the collection unit accumulates one week to one month of the user's past health data (e.g., heart rate time-series vector, stress level score, exercise history, sleep pattern, etc.) as time-series tensors and inputs them into an AI analysis module. The collection unit first performs preprocessing such as missing value imputation, outlier removal, and time-series normalization on the input data. For anomaly detection, the collection unit uses autoencoders, time-series clustering algorithms (e.g., k-means, DBSCAN), or LSTM-based anomaly prediction models. Examples of AI inputs include (1) heart rate time-series for the past 30 days (1,440 elements/day×30 days), (2) daily stress level scores (30 elements), and (3) exercise history vectors (e.g., weekly exercise intensity, calories burned, exercise type array). The collection unit generates outputs such as (a) anomaly pattern detection label (e.g., “Heart rate anomaly: Yes”), (b) recommended collection frequency (e.g., “Heart rate: every 5 minutes,” “Stress: every hour”), and (c) recovery status evaluation (e.g., “Delayed recovery,” “Normal recovery”). Example outputs include “Anomaly detected: high heart rate,” “Recommended collection frequency: every minute,” “Recovery status: delayed.” Based on these outputs, the collection scheduler module automatically adjusts the data acquisition interval for each sensor, increasing collection frequency during periods when anomalies are predicted or during recovery after exercise. Internally, the AI model uses reconstruction error or cross-entropy loss as the loss function and updates weights by gradient descent to continuously improve the accuracy of anomaly detection and pattern prediction. For individual optimization for each user, transfer learning and online learning are applied, and the user's past data and feedback are sequentially reflected in learning. Unlike conventional empirical rules and fixed collection schedules by humans, the collection unit achieves optimal data collection responsive to individual health conditions and life rhythms through high-dimensional time-series analysis and real-time control by AI. Application fields include health management for general consumers, monitoring of chronic disease patients, recovery management for athletes, and remote monitoring in medical institutions. Thus, the present invention achieves not only automation of human tasks but also essential improvement of computer technology for personalized data collection optimization by AI.

The collection unit can perform filtering based on the user's current activity status or environment when collecting health data. For example, if the user is exercising, the collection unit preferentially collects data related to exercise (heart rate, calories burned, etc.). The collection unit may use AI to determine the user's current activity status or environment. For example, the collection unit detects the user's activity status using sensors and analyzes the data to identify the activity status. The collection unit may also collect data related to relaxation (heart rate variability, stress level, etc.) if the user is resting. For example, the collection unit analyzes heart rate variability to evaluate relaxation status. Furthermore, if the user is outdoors, the collection unit collects external environmental data such as ambient noise and temperature and evaluates their impact on health condition. For example, the collection unit analyzes ambient noise to evaluate noise level. By performing filtering based on the user's current activity status or environment, the collection unit can collect highly relevant data. Some or all of the above-described processing in the collection unit may be performed using AI or without using AI. For example, the collection unit may input the user's activity status data into a generative AI and have the generative AI perform filtering. Specifically, the collection unit acquires multidimensional data from accelerometers, gyroscopes, GPS, and environmental sensors (e.g., temperature, humidity, noise level) as time-series tensors. The collection unit first performs noise removal and normalization (e.g., low-pass filtering of acceleration, Z-score normalization of environmental data) on these data. For activity status estimation, the collection unit uses convolutional neural networks (CNN), recurrent neural networks (RNN), or Transformer-based time-series classification models. Examples of AI inputs include (1) 10 seconds of triaxial acceleration data (3 dimensions×100 samples), (2) GPS location information (latitude, longitude, altitude as continuous values), (3) environmental sound spectrum (e.g., FFT feature vector per second), and (4) time-series data of temperature and humidity. The collection unit generates outputs such as (a) activity label (e.g., “Exercising,” “Resting,” “Outdoors”), (b) environment state label (e.g., “High noise,” “High temperature”), and (c) recommended data types to collect (e.g., “Prioritize heart rate,” “Prioritize stress level”). Example outputs include “Activity: Exercising,” “Environment: High noise,” “Recommended collection: heart rate, calories burned.” Based on these outputs, the collection control module dynamically switches the data acquisition targets and frequency for each sensor, prioritizing heart rate and calories burned during exercise, heart rate variability and stress level during rest, and ambient noise and temperature during outdoor activities. Internally, the AI model uses cross-entropy loss or mean squared error to continuously improve activity recognition accuracy, and applies transfer learning or online learning using user feedback and past activity history. Unlike conventional subjective activity records and fixed data collection by humans, the collection unit achieves real-time and high-precision activity and environment recognition and data collection optimization through multidimensional data analysis and automatic control by AI. Application fields include health management for general consumers, training monitoring for athletes, safety management at work sites, and lifestyle disease management in medical institutions. Thus, the present invention achieves not only automation of human tasks but also essential improvement of computer technology for activity and environment recognition and data collection optimization by AI.

The collection unit can estimate the user's emotion and determine a priority of health data to be collected based on the estimated emotion of the user. For example, if the user is feeling stressed, the collection unit preferentially collects data related to stress level (heart rate, skin electrical activity, etc.). The collection unit uses emotion estimation functions, such as an emotion engine or generative AI, to estimate the user's emotion. For example, the collection unit estimates the user's emotion using facial recognition technology. Facial recognition technology analyzes facial data captured by a camera to estimate emotion. The collection unit may also estimate the user's emotion using voice analysis technology. Voice analysis technology analyzes the tone and speed of the user's voice to estimate emotion. Furthermore, if the user is relaxed, the collection unit preferentially collects data related to relaxation (heart rate variability, respiratory rate, etc.). For example, if the user is exercising, the collection unit preferentially collects data related to exercise (calories burned, exercise intensity, etc.). By determining a priority of health data to be collected based on the user's emotion, the collection unit can preferentially collect important data. Some or all of the above-described processing in the collection unit may be performed using AI or without using AI. For example, the collection unit may input the user's emotion data into a generative AI and have the generative AI determine the priority of health data to be collected. Specifically, the collection unit acquires multidimensional data for emotion estimation, such as facial expression feature vectors (e.g., 68 dimensions), voice features (e.g., MFCC, pitch, speech rate), and biosignals (e.g., heart rate, skin electrical activity, respiratory rate) as time-series tensors, and applies noise removal and normalization before inputting them into convolutional neural networks (CNN), recurrent neural networks (RNN), or Transformer-based multimodal emotion recognition models. Examples of AI inputs include (1) facial expression feature vectors per second (68 dimensions×30 frames), (2) 3 seconds of voice MFCC features (20 dimensions×30 frames), (3) 30 seconds of heart rate time-series (30 elements), (4) continuous skin electrical activity values (60 elements), and (5) respiratory rate time-series (30 elements). The collection unit generates outputs such as (a) emotion label (e.g., “Stress,” “Relaxation”), (b) emotion score (e.g., 0 to 100), and (c) recommended data priority list (e.g., “Heart rate>Skin electrical activity>Respiratory rate”). Example outputs include “Emotion: Stress,” “Priority collection: heart rate, skin electrical activity,” “Emotion: Relaxation,” “Priority collection: heart rate variability, respiratory rate.” Based on these outputs, the collection control module dynamically switches the data acquisition priority for each sensor, focusing on stress-related data during stress, relaxation-related data during relaxation, and exercise-related data during exercise. Internally, the AI model uses cross-entropy loss or mean squared error to continuously improve emotion estimation accuracy, and applies transfer learning or online learning using user feedback and past emotion history. Unlike conventional subjective judgment and fixed data collection by humans, the collection unit achieves real-time and high-precision emotion recognition and data priority control through multidimensional data analysis and automatic control by AI. Application fields include health management for general consumers, stress monitoring, psychological state management for athletes, and mental health support in medical institutions. Thus, the present invention achieves not only automation of human tasks but also essential improvement of computer technology for emotion recognition and data collection priority optimization by AI.

The collection unit can consider a geographic location information of the user when collecting health data and preferentially collect highly relevant data. For example, if the user is at a high altitude, the collection unit preferentially collects oxygen saturation and heart rate data. The collection unit may use AI to consider the user's geographic location information. For example, the collection unit obtains the user's location information using a GPS sensor and analyzes the data to identify the location. The collection unit may also preferentially collect ambient noise and air pollution level data if the user is in an urban area. For example, the collection unit analyzes ambient noise to evaluate noise level. Furthermore, if the user is in a natural environment, the collection unit preferentially collects environmental data such as temperature and humidity. For example, the collection unit collects environmental data using temperature and humidity sensors. By considering the user's geographic location information, the collection unit can preferentially collect highly relevant data. Some or all of the above-described processing in the collection unit may be performed using AI or without using AI. For example, the collection unit may input the user's location information data into a generative AI and have the generative AI collect highly relevant data. Specifically, the collection unit acquires multidimensional data such as latitude, longitude, and altitude from GPS sensors, barometric pressure sensors, and environmental sensors (e.g., temperature, humidity, noise, air pollution level) as time-series tensors. The collection unit first performs noise removal and normalization (e.g., smoothing of location data, Z-score normalization of environmental data) on these data. For estimating the relationship between geographic location information and health risk, the collection unit uses machine learning algorithms (e.g., decision trees, random forests, Transformer-based time-series classification models). Examples of AI inputs include (1) current latitude, longitude, and altitude (3 dimensions), (2) movement trajectory for the past hour (60 samples), (3) time-series data of temperature, humidity, and barometric pressure (each 60 elements), and (4) noise level and air pollution index (each 60 elements). The collection unit generates outputs such as (a) current location category (e.g., “High altitude,” “Urban area,” “Natural environment”), (b) recommended data types to collect (e.g., “Oxygen saturation, heart rate,” “Noise, air pollution,” “Temperature, humidity”), and (c) health risk evaluation (e.g., “High altitude risk: high,” “Urban area noise: high”). Example outputs include “Current location: high altitude,” “Recommended collection: oxygen saturation, heart rate,” “Health risk: high.” Based on these outputs, the collection control module dynamically switches the data acquisition targets and frequency for each sensor, focusing on oxygen saturation and heart rate at high altitude, noise and air pollution in urban areas, and temperature and humidity in natural environments. Internally, the AI model uses cross-entropy loss or mean squared error to continuously improve location recognition and risk estimation accuracy, and applies transfer learning or online learning using user movement history and changes in health condition. Unlike conventional subjective location recognition and fixed data collection by humans, the collection unit achieves real-time and high-precision geographic risk recognition and data collection optimization through multidimensional data analysis and automatic control by AI. Application fields include health management for mountaineers and high-altitude residents, environmental risk monitoring for urban dwellers, health management for travelers, and remote monitoring in medical institutions. Thus, the present invention achieves not only automation of human tasks but also essential improvement of computer technology for geographic location recognition and data collection optimization by AI.

The collection unit can analyze a user's social media activity when collecting health data and collect relevant data. For example, if the user posts about feeling stressed on social media, the collection unit preferentially collects data related to stress level. The collection unit may use AI to analyze the user's social media activity. For example, the collection unit analyzes the content of social media posts using natural language processing technology to estimate emotion. The collection unit may also preferentially collect data related to exercise if the user posts about exercise. For example, the collection unit analyzes the content of exercise-related posts to identify the type and intensity of exercise. Furthermore, the collection unit may preferentially collect data related to nutritional balance if the user posts about meals. For example, the collection unit analyzes the content of meal-related posts to identify the type and amount of food. By analyzing the user's social media activity, the collection unit can collect relevant data. Some or all of the above-described processing in the collection unit may be performed using AI or without using AI. For example, the collection unit may input social media post data into a generative AI and have the generative AI collect relevant data. Specifically, the collection unit inputs the user's social media post data (e.g., text, images, posting time, hashtags, etc.) into a natural language processing module or image analysis module. For text data, the collection unit performs morphological analysis, word embedding (Word2Vec, BERT, etc.), sentiment analysis (e.g., labeling as positive, negative, stress, etc.), and topic classification (e.g., exercise, diet, stress, etc.). For image data, the collection unit uses a CNN-based image classification model to estimate the content of exercise or meals. Examples of AI inputs include (1) post text (e.g., “I'm tired from work today,” “Achieved 5 km run”), (2) post images (e.g., meal photos, exercise selfies), (3) posting time and frequency data (e.g., number of posts per day), and (4) hashtags (e.g., “# stress,” “# workout”). The collection unit generates outputs such as (a) emotion label (e.g., “Stress,” “Sense of achievement”), (b) topic label (e.g., “Exercise,” “Diet”), and (c) recommended data types to collect (e.g., “Stress level,” “Exercise intensity,” “Meal content”). Example outputs include “Emotion: Stress,” “Recommended collection: stress level,” “Topic: Exercise,” “Recommended collection: exercise intensity.” Based on these outputs, the collection control module dynamically switches the data acquisition targets and frequency for each sensor, focusing on stress-related data during stress posts, exercise-related data during exercise posts, and meal-related data during meal posts. Internally, the AI model uses cross-entropy loss or mean squared error to continuously improve emotion and topic estimation accuracy, and applies transfer learning or online learning using user feedback and past post history. Unlike conventional subjective interpretation of posts and fixed data collection by humans, the collection unit achieves real-time and high-precision social media analysis and data collection optimization through multidimensional data analysis and automatic control by AI. Application fields include health management for general consumers, stress monitoring, training management for athletes, and lifestyle disease support in medical institutions. Thus, the present invention achieves not only automation of human tasks but also essential improvement of computer technology for social media analysis and data collection optimization by AI.

The analysis unit can estimate the user's emotion and adjust a method of expressing analysis based on the estimated emotion of the user. For example, if the user is feeling stressed, the analysis unit provides a simple and highly visible analysis result. The analysis unit uses emotion estimation functions, such as an emotion engine or generative AI, to estimate the user's emotion. For example, the analysis unit estimates the user's emotion using facial recognition technology. Facial recognition technology analyzes facial data captured by a camera to estimate emotion. The analysis unit may also estimate the user's emotion using voice analysis technology. Voice analysis technology analyzes the tone and speed of the user's voice to estimate emotion. Furthermore, if the user is relaxed, the analysis unit provides detailed analysis results to enable deeper understanding. For example, if the user is in a hurry, the analysis unit provides concise analysis results focusing on key points. By adjusting a method of expressing analysis based on the user's emotion, the analysis unit can provide analysis results that are easy for the user to understand. Some or all of the above-described processing in the analysis unit may be performed using AI or without using AI. For example, the analysis unit may input the user's emotion data into a generative AI and have the generative AI adjust a method of expressing analysis. Specifically, the analysis unit acquires multidimensional data for emotion estimation, such as facial expression feature vectors (e.g., 68 dimensions), voice features (e.g., MFCC, pitch, speech rate), and biosignals (e.g., heart rate, skin electrical activity) as time-series tensors, and applies noise removal and normalization (e.g., face image alignment, voice normalization, Z-score normalization of time-series data) before inputting them into convolutional neural networks (CNN), recurrent neural networks (RNN), or Transformer-based multimodal emotion recognition models. Examples of AI inputs include (1) facial expression feature vectors per second (68 dimensions×30 frames), (2) 3 seconds of voice MFCC features (20 dimensions×30 frames), and (3) 30 seconds of heart rate time-series (30 elements). The analysis unit generates outputs such as (a) emotion label (e.g., “Stress,” “Relaxation”), (b) emotion score (e.g., 0 to 100), and (c) emotion trend (e.g., “Stress increasing”). Example outputs include “Emotion: Stress,” “Emotion score: 85,” “Emotion trend: Increasing.” Based on these outputs, the analysis unit passes data to the analysis result expression control module, and dynamically switches the method of expressing analysis results according to the user's psychological state and situation, such as “simple graph display emphasizing key points” during stress, “detailed explanation with numerical values and graphs” during relaxation, and “summary in bullet points” when in a hurry. Internally, the AI model uses cross-entropy loss or mean squared error to continuously improve emotion estimation and expression optimization accuracy, and applies transfer learning or online learning using user feedback and past emotion and comprehension history. Unlike conventional subjective judgment and fixed analysis result display by humans, the analysis unit achieves optimal analysis result expression responsive to the user's psychological state and situation through high-dimensional multimodal data analysis and real-time control by AI. Application fields include health management for general consumers, stress monitoring, psychological state management for athletes, and mental health support in medical institutions. Thus, the present invention achieves not only automation of human tasks but also essential improvement of computer technology for emotion recognition and analysis result expression optimization by AI.

The analysis unit can adjust a level of detail of analysis based on an importance of health data during analysis. For example, if heart rate data is abnormal, the analysis unit performs detailed analysis to identify the cause of the abnormality. The analysis unit may use AI to evaluate the importance of health data. For example, the analysis unit evaluates the importance of data using machine learning algorithms and performs detailed analysis for important data. The analysis unit may also perform detailed analysis to identify the cause of stress if the stress level is high. For example, the analysis unit analyzes fluctuations in stress level to identify the cause of stress. Furthermore, if exercise data is within the normal range, the analysis unit performs concise analysis and provides feedback to the user. For example, the analysis unit calculates the mean and standard deviation of exercise data and provides concise feedback. By adjusting a level of detail of analysis based on the importance of health data, the analysis unit can perform detailed analysis for important data. Some or all of the above-described processing in the analysis unit may be performed using AI or without using AI. For example, the analysis unit may input health data into a generative AI and have the generative AI adjust a level of detail of analysis. Specifically, the analysis unit acquires multidimensional health data such as heart rate time-series data (e.g., 30-element vector per second), stress level score (e.g., continuous value from 0 to 100), exercise data (e.g., acceleration vector, calories burned), and blood pressure data (e.g., continuous value for 10 minutes) as time-series tensors, and applies noise removal and normalization (e.g., Z-score normalization, outlier removal) before inputting them into convolutional neural networks (CNN), recurrent neural networks (RNN), or Transformer-based time-series analysis models. Examples of AI inputs include (1) 30 seconds of heart rate time-series (30 elements), (2) one minute of stress level score (60 elements), (3) exercise intensity vector (10 elements), and (4) continuous blood pressure values (60 elements). The analysis unit generates outputs such as (a) importance score (e.g., continuous value from 0 to 1), (b) anomaly detection label (e.g., “Heart rate anomaly: Yes”), and (c) recommended analysis detail level (e.g., “Detailed analysis,” “Simple analysis”). Example outputs include “Importance: 0.92,” “Recommended analysis: detailed,” “Cause of anomaly: increased exercise load,” “Importance: 0.15,” “Recommended analysis: simple.” Based on these outputs, the analysis detail control module dynamically switches the analysis algorithm and output granularity for each data type, performing detailed time-series analysis and cause estimation during anomalies and summary display of mean and standard deviation during normal conditions. Internally, the AI model uses mean squared error or cross-entropy loss as the loss function and updates weights by gradient descent to continuously improve importance estimation and cause identification accuracy. For individual optimization for each user, transfer learning and online learning are applied, and the user's past data and feedback are sequentially reflected in learning. Unlike conventional empirical rules and uniform analysis granularity by humans, the analysis unit achieves optimal analysis detail responsive to individual health conditions and data importance through high-dimensional time-series analysis and automatic control by AI. Application fields include health management for general consumers, monitoring of chronic disease patients, recovery management for athletes, and remote monitoring in medical institutions. Thus, the present invention achieves not only automation of human tasks but also essential improvement of computer technology for data importance recognition and analysis detail optimization by AI.

The analysis unit can apply different analysis algorithms according to a category of health data during analysis. For example, the analysis unit applies a heart rate variability analysis algorithm to heart rate data. The analysis unit may use AI to identify the category of health data. For example, the analysis unit classifies the category of data using machine learning algorithms and applies appropriate analysis algorithms. The analysis unit may also apply a skin electrical activity analysis algorithm to stress level data. For example, the analysis unit analyzes skin electrical activity data to evaluate stress level. Furthermore, the analysis unit may apply a calorie consumption calculation algorithm to exercise data. For example, the analysis unit analyzes exercise data to calculate calories burned. By applying different analysis algorithms according to a category of health data, the analysis unit can perform optimal analysis for each data type. Some or all of the above-described processing in the analysis unit may be performed using AI or without using AI. For example, the analysis unit may input health data into a generative AI and have the generative AI apply analysis algorithms. Specifically, the analysis unit acquires multidimensional health data such as heart rate time-series data (e.g., 30-element vector per second), skin electrical activity data (e.g., 60 continuous values per minute), exercise data (e.g., acceleration vector, calories burned), blood pressure data (e.g., continuous value for 10 minutes), and meal record text (e.g., “Lunch: salad, chicken, rice”) as time-series tensors or text, and applies noise removal and normalization (e.g., Z-score normalization, outlier removal, text preprocessing) before inputting them into convolutional neural networks (CNN), recurrent neural networks (RNN), Transformer-based time-series analysis models, or natural language processing models. Examples of AI inputs include (1) 30 seconds of heart rate time-series (30 elements), (2) one minute of continuous skin electrical activity values (60 elements), (3) exercise intensity vector (10 elements), (4) continuous blood pressure values (60 elements), and (5) meal record text. The analysis unit generates outputs such as (a) data category label (e.g., “Heart rate,” “Stress,” “Exercise,” “Diet”), (b) recommended analysis algorithm (e.g., “Heart rate variability analysis,” “Skin electrical activity analysis,” “Calorie consumption calculation,” “Nutrient extraction”), and (c) analysis result (e.g., “Heart rate variability: normal,” “Stress level: high,” “Calories burned: 250 kcal,” “Nutrition balance: excess fat”). Example outputs include “Category: heart rate,” “Analysis algorithm: heart rate variability analysis,” “Result: normal.” Based on these outputs, the analysis algorithm selection module automatically selects and applies the optimal analysis method for each data type and generates analysis results. Internally, the AI model uses mean squared error or cross-entropy loss as the loss function and updates weights by gradient descent to continuously improve category classification and analysis accuracy. For individual optimization for each user, transfer learning and online learning are applied, and the user's past data and feedback are sequentially reflected in learning. Unlike conventional empirical rules and uniform analysis methods by humans, the analysis unit achieves optimal application of analysis algorithms for each data type through high-dimensional data analysis and automatic control by AI. Application fields include health management for general consumers, monitoring of chronic disease patients, recovery management for athletes, and remote monitoring in medical institutions. Thus, the present invention achieves not only automation of human tasks but also essential improvement of computer technology for data category recognition and analysis algorithm optimization by AI.

The analysis unit can estimate the user's emotion and adjust a length of analysis based on the estimated emotion of the user. For example, if the user is feeling stressed, the analysis unit provides a short analysis result focusing on key points. The analysis unit uses emotion estimation functions, such as an emotion engine or generative AI, to estimate the user's emotion. For example, the analysis unit estimates the user's emotion using facial recognition technology. Facial recognition technology analyzes facial data captured by a camera to estimate emotion. The analysis unit may also estimate the user's emotion using voice analysis technology. Voice analysis technology analyzes the tone and speed of the user's voice to estimate emotion. Furthermore, if the user is relaxed, the analysis unit provides detailed analysis results to enable deeper understanding. For example, if the user is in a hurry, the analysis unit provides concise analysis results. By adjusting a length of analysis based on the user's emotion, the analysis unit can provide analysis results of appropriate length for the user. Some or all of the above-described processing in the analysis unit may be performed using AI or without using AI. For example, the analysis unit may input the user's emotion data into a generative AI and have the generative AI adjust a length of analysis. Specifically, the analysis unit acquires multidimensional data for emotion estimation, such as facial expression feature vectors (e.g., 68 dimensions), voice features (e.g., MFCC, pitch, speech rate), and biosignals (e.g., heart rate, skin electrical activity) as time-series tensors, and applies noise removal and normalization (e.g., face image alignment, voice normalization, Z-score normalization of time-series data) before inputting them into convolutional neural networks (CNN), recurrent neural networks (RNN), or Transformer-based multimodal emotion recognition models. Examples of AI inputs include (1) facial expression feature vectors per second (68 dimensions×30 frames), (2) 3 seconds of voice MFCC features (20 dimensions×30 frames), and (3) 30 seconds of heart rate time-series (30 elements). The analysis unit generates outputs such as (a) emotion label (e.g., “Stress,” “Relaxation”), (b) emotion score (e.g., 0 to 100), and (c) recommended analysis length (e.g., “Shortened,” “Detailed,” “Summary”). Example outputs include “Emotion: Stress,” “Recommended length: shortened,” “Emotion: Relaxation,” “Recommended length: detailed.” Based on these outputs, the analysis result generation module dynamically switches the length and detail of analysis content according to the user's psychological state and situation, such as “short text display focusing only on key points” during stress, “detailed explanation display” during relaxation, and “summary in bullet points” when in a hurry. Internally, the AI model uses cross-entropy loss or mean squared error to continuously improve emotion estimation and expression optimization accuracy, and applies transfer learning or online learning using user feedback and past emotion and comprehension history. Unlike conventional subjective judgment and fixed analysis result display by humans, the analysis unit achieves optimal analysis result length responsive to the user's psychological state and situation through high-dimensional multimodal data analysis and real-time control by AI. Application fields include health management for general consumers, stress monitoring, psychological state management for athletes, and mental health support in medical institutions. Thus, the present invention achieves not only automation of human tasks but also essential improvement of computer technology for emotion recognition and analysis result length optimization by AI.

The analysis unit can determine a priority of analysis based on a timing of health data collection during analysis. For example, the analysis unit preferentially analyzes recently collected data and provides real-time feedback. The analysis unit may use AI to evaluate the timing of health data collection. For example, the analysis unit analyzes the timing of data collection using machine learning algorithms and determines the priority. The analysis unit may also analyze past data to evaluate long-term health trends. For example, the analysis unit predicts health trends based on past data and provides feedback. Furthermore, the analysis unit may preferentially analyze data collected immediately after specific events (exercise, meals, etc.). For example, the analysis unit analyzes data after exercise to evaluate the effect of exercise. By determining a priority of analysis based on the timing of health data collection, the analysis unit can provide real-time feedback. Some or all of the above-described processing in the analysis unit may be performed using AI or without using AI. For example, the analysis unit may input health data into a generative AI and have the generative AI determine a priority of analysis. Specifically, the analysis unit acquires health data collection time information (e.g., timestamped heart rate vector, stress level score, exercise history, meal record, etc.) as time-series tensors, and applies noise removal and normalization (e.g., time normalization, outlier removal) before inputting them into convolutional neural networks (CNN), recurrent neural networks (RNN), or Transformer-based time-series analysis models. Examples of AI inputs include (1) heart rate time-series for the past 24 hours (1,440 elements), (2) daily stress level score (30 elements), (3) exercise event timestamps (e.g., exercise start and end times), and (4) timestamped meal record text. The analysis unit generates outputs such as (a) analysis priority list (e.g., “Latest data >Post-exercise data>Past data”), (b) health trend evaluation (e.g., “Heart rate increasing trend,” “Stress decreasing trend”), and (c) recommended real-time feedback (e.g., “Immediate analysis recommended”). Example outputs include “Priority analysis: latest heart rate,” “Trend: stress decreasing,” “Feedback: immediate.” Based on these outputs, the analysis scheduler module dynamically switches the analysis order and timing for each data type, preferentially analyzing latest data and post-event data and providing real-time feedback to the user. Internally, the AI model uses mean squared error or cross-entropy loss as the loss function and updates weights by gradient descent to continuously improve priority estimation and trend evaluation accuracy. For individual optimization for each user, transfer learning and online learning are applied, and the user's past data and feedback are sequentially reflected in learning. Unlike conventional empirical rules and uniform analysis order by humans, the analysis unit achieves optimal analysis priority responsive to individual health conditions and events through high-dimensional time-series analysis and automatic control by AI. Application fields include health management for general consumers, monitoring of chronic disease patients, recovery management for athletes, and remote monitoring in medical institutions. Thus, the present invention achieves not only automation of human tasks but also essential improvement of computer technology for data collection timing recognition and analysis priority optimization by AI.

The analysis unit can adjust an order of analysis based on a relevance of health data during analysis. For example, the analysis unit evaluates the relevance between heart rate data and stress level data and preferentially analyzes highly relevant data. The analysis unit may use AI to evaluate the relevance of health data. For example, the analysis unit analyzes the relevance of data using machine learning algorithms and adjusts the order of analysis. The analysis unit may also evaluate the relevance between exercise data and calorie consumption data and preferentially analyze highly relevant data. For example, the analysis unit analyzes the correlation between exercise data and calorie consumption data and adjusts the order of analysis. Furthermore, the analysis unit may evaluate the relevance between meal data and nutrition balance data and preferentially analyze highly relevant data. For example, the analysis unit analyzes the relevance between meal data and nutrition balance data and adjusts the order of analysis. By adjusting the order of analysis based on the relevance of health data, the analysis unit can preferentially analyze highly relevant data. Some or all of the above-described processing in the analysis unit may be performed using AI or without using AI. For example, the analysis unit may input health data into a generative AI and have the generative AI adjust the order of analysis. Specifically, the analysis unit acquires multidimensional health data such as heart rate time-series data (e.g., 30-element vector per second), stress level score (e.g., continuous value from 0 to 100), exercise data (e.g., acceleration vector, calories burned), meal record text (e.g., “Lunch: salad, chicken, rice”), and nutrition balance data (e.g., intake amount vector for each nutrient) as time-series tensors or text, and applies noise removal and normalization (e.g., Z-score normalization, outlier removal, text preprocessing) before inputting them into convolutional neural networks (CNN), recurrent neural networks (RNN), Transformer-based time-series analysis models, or natural language processing models. Examples of AI inputs include (1) 30 seconds of heart rate time-series (30 elements), (2) one minute of stress level score (60 elements), (3) exercise intensity vector (10 elements), (4) calorie consumption time-series (10 elements), (5) meal record text, and (6) nutrient intake amount vector (10 elements). The analysis unit generates outputs such as (a) data relevance score (e.g., continuous value from 0 to 1), (b) analysis order list (e.g., “Heart rate→Stress→Exercise→Calorie consumption”), and (c) recommended analysis pairs (e.g., “Exercise & Calorie consumption,” “Meal & Nutrition balance”). Example outputs include “Relevance: 0.85 (heart rate & stress),” “Priority analysis: heart rate & stress,” “Relevance: 0.92 (exercise & calorie consumption).” Based on these outputs, the analysis order control module dynamically switches the analysis order and pairing for each data type, preferentially analyzing highly relevant data and realizing comprehensive health condition evaluation and feedback. Internally, the AI model uses mean squared error or cross-entropy loss as the loss function and updates weights by gradient descent to continuously improve relevance estimation and analysis order optimization accuracy. For individual optimization for each user, transfer learning and online learning are applied, and the user's past data and feedback are sequentially reflected in learning. Unlike conventional empirical rules and uniform analysis order by humans, the analysis unit achieves optimal analysis order responsive to data relevance through high-dimensional data analysis and automatic control by AI. Application fields include health management for general consumers, monitoring of chronic disease patients, recovery management for athletes, and remote monitoring in medical institutions. Thus, the present invention achieves not only automation of human tasks but also essential improvement of computer technology for data relevance recognition and analysis order optimization by AI.

The generation unit can estimate the user's emotion and adjust a method of expressing advice based on the estimated emotion of the user. For example, if the user is feeling stressed, the generation unit provides simple and highly visible advice. The generation unit uses emotion estimation functions, such as an emotion engine or generative AI, to estimate the user's emotion. For example, the generation unit estimates the user's emotion using facial recognition technology. Facial recognition technology analyzes facial data captured by a camera to estimate emotion. The generation unit may also estimate the user's emotion using voice analysis technology. Voice analysis technology analyzes the tone and speed of the user's voice to estimate emotion. Furthermore, if the user is relaxed, the generation unit provides detailed advice to enable deeper understanding. For example, if the user is in a hurry, the generation unit provides concise advice focusing on key points. By adjusting a method of expressing advice based on the user's emotion, the generation unit can provide advice that is easy for the user to understand. Some or all of the above-described processing in the generation unit may be performed using AI or without using AI. For example, the generation unit may input the user's emotion data into a generative AI and have the generative AI adjust a method of expressing advice. Specifically, the generation unit acquires multidimensional data for emotion estimation, such as facial expression feature vectors (e.g., 68 dimensions), voice features (e.g., MFCC, pitch, speech rate), and biosignals (e.g., heart rate, skin electrical activity) as time-series tensors, and applies noise removal and normalization (e.g., face image alignment, voice normalization, Z-score normalization of time-series data) before inputting them into convolutional neural networks (CNN), recurrent neural networks (RNN), or Transformer-based multimodal emotion recognition models. Examples of AI inputs include (1) facial expression feature vectors per second (68 dimensions×30 frames), (2) 3 seconds of voice MFCC features (20 dimensions×30 frames), and (3) 30 seconds of heart rate time-series (30 elements). The generation unit generates outputs such as (a) emotion label (e.g., “Stress,” “Relaxation,” “Anger”), (b) emotion score (e.g., continuous value from 0 to 100), and (c) emotion trend (e.g., “Stress increasing”). Example outputs include “Emotion: Stress,” “Emotion score: 85,” “Emotion trend: Increasing.” Based on these outputs, the generation unit passes data to the advice expression control module, and dynamically switches the method of expressing advice according to the user's psychological state and situation, such as “simple text display emphasizing key points” during stress, “detailed explanation display” during relaxation, and “summary in bullet points” when in a hurry. Internally, the AI model uses cross-entropy loss or mean squared error to continuously improve emotion estimation and expression optimization accuracy, and applies transfer learning or online learning using user feedback and past emotion and comprehension history. Unlike conventional subjective judgment and fixed advice display by humans, the generation unit achieves optimal advice expression responsive to the user's psychological state and situation through high-dimensional multimodal data analysis and real-time control by AI. Application fields include health management for general consumers, stress monitoring, psychological state management for athletes, and mental health support in medical institutions. Thus, the present invention achieves not only automation of human tasks but also essential improvement of computer technology for emotion recognition and advice expression optimization by AI.

The generation unit can adjust the level of detail of advice based on the importance of the health condition when generating advice. For example, when the heart rate is abnormal, the generation unit provides detailed advice and explains the cause and countermeasures of the abnormality. The generation unit can use AI to evaluate the importance of the health condition. For instance, the generation unit may use machine learning algorithms to assess the importance of data and provide detailed advice for significant health conditions. Additionally, when the stress level is high, the generation unit can provide detailed advice and explain the causes and countermeasures of stress. For example, the generation unit analyzes fluctuations in stress level, identifies the cause of stress, and proposes countermeasures. Furthermore, when the health condition is normal, the generation unit can provide concise advice and feedback to the user. For example, the generation unit calculates the average and standard deviation of health conditions and provides concise feedback. By adjusting the level of detail of advice based on the importance of the health condition, detailed advice can be provided for significant health conditions. Some or all of the above-described processes in the generation unit may be performed using AI or without using AI. For example, the generation unit may input health condition data into a generation AI and have the generation AI adjust the level of detail of advice. Specifically, the generation unit acquires multidimensional health data such as heart rate time series data (e.g., 30-element vector per second), stress level scores (e.g., continuous values from 0 to 100), exercise data (e.g., acceleration vectors, calories burned), and blood pressure data (e.g., continuous values over 10 minutes) as time series tensors, applies noise removal and normalization (e.g., Z-score normalization, outlier removal), and inputs them into time series analysis models based on convolutional neural networks (CNN), recurrent neural networks (RNN), or Transformers. Example inputs to the AI include (1) 30 seconds of heart rate time series (30 elements), (2) 1 minute of stress level scores (60 elements), (3) exercise intensity vectors (10 elements), and (4) blood pressure continuous values (60 elements). From these inputs, the generation unit produces outputs such as (a) importance scores (e.g., continuous values from 0 to 1), (b) anomaly detection labels (e.g., “heart rate abnormal: yes”), and (c) recommended advice detail level (e.g., “detailed advice,” “simple advice”). Example outputs include “importance: 0.92,” “recommended advice: detailed,” “cause of anomaly: increased exercise load,” “importance: 0.15,” “recommended advice: simple.” Based on these outputs, the advice detail control module dynamically switches the advice generation algorithm and output granularity for each data type, performing detailed time series analysis and cause estimation in case of anomalies, and summary display of averages and standard deviations in case of normal conditions. The generation unit uses mean squared error or cross-entropy loss as the loss function inside the AI model and updates weights via gradient descent to continuously improve the accuracy of importance estimation and anomaly cause identification. For individual optimization per user, the generation unit applies transfer learning or online learning, sequentially reflecting the user's past data and feedback in the learning process. The generation unit achieves technical effects of optimizing advice detail level in response to individual health conditions and data importance, which was difficult with conventional human heuristics or uniform advice granularity, through high-dimensional time series analysis and automatic control by AI. Application fields include health management for general consumers, monitoring of chronic disease patients, recovery management for athletes, and remote monitoring in medical institutions. Thus, the present invention not only automates human tasks but also realizes essential improvements in computer technology, such as AI-based recognition of data importance and optimization of advice detail level.

The generation unit can apply different advice algorithms according to the category of health condition when generating advice. For example, for heart rate data, the generation unit applies a heart rate variability analysis algorithm to generate appropriate advice. The generation unit can use AI to identify the category of health condition. For instance, the generation unit may use machine learning algorithms to classify data categories and apply suitable advice algorithms. Additionally, for stress level data, the generation unit can apply a skin conductance activity analysis algorithm to generate appropriate advice. For example, the generation unit analyzes skin conductance activity data, evaluates stress levels, and provides appropriate advice. Furthermore, for exercise data, the generation unit can apply a calorie consumption calculation algorithm to generate appropriate advice. For example, the generation unit analyzes exercise data, calculates calories burned, and provides appropriate advice. By applying different advice algorithms according to the category of health condition, optimal advice can be provided. Some or all of the above-described processes in the generation unit may be performed using AI or without using AI. For example, the generation unit may input health condition data into a generation AI and have the generation AI apply advice algorithms. Specifically, the generation unit acquires multidimensional health data such as heart rate time series data (e.g., 30-element vector per second), skin conductance activity data (e.g., 60-element continuous values per minute), exercise data (e.g., acceleration vectors, calories burned), blood pressure data (e.g., continuous values over 10 minutes), and meal record texts (e.g., “Lunch: salad, chicken, rice”) as time series tensors or text, applies noise removal and normalization (e.g., Z-score normalization, outlier removal, text preprocessing), and inputs them into time series analysis models based on CNN, RNN, Transformer, or natural language processing models. Example inputs to the AI include (1) 30 seconds of heart rate time series (30 elements), (2) 1 minute of skin conductance activity continuous values (60 elements), (3) exercise intensity vectors (10 elements), (4) blood pressure continuous values (60 elements), and (5) meal record texts. From these inputs, the generation unit produces outputs such as (a) data category labels (e.g., “heart rate,” “stress,” “exercise,” “meal”), (b) recommended advice algorithms (e.g., “heart rate variability analysis,” “skin conductance activity analysis,” “calorie consumption calculation,” “nutrient extraction”), and (c) advice content (e.g., “heart rate variability: normal,” “stress level: high,” “calories burned: 250 kcal,” “nutritional balance: excessive fat”). Example outputs include “category: heart rate,” “advice algorithm: heart rate variability analysis,” “content: normal.” Based on these outputs, the advice algorithm selection module automatically selects and applies the optimal advice generation method for each data type and generates advice content. The generation unit uses mean squared error or cross-entropy loss as the loss function inside the AI model and updates weights via gradient descent to continuously improve the accuracy of category classification and advice. For individual optimization per user, the generation unit applies transfer learning or online learning, sequentially reflecting the user's past data and feedback in the learning process. The generation unit achieves technical effects of optimal advice algorithm application for each data type, which was difficult with conventional human heuristics or uniform advice methods, through high-dimensional data analysis and automatic control by AI. Application fields include health management for general consumers, monitoring of chronic disease patients, recovery management for athletes, and remote monitoring in medical institutions. Thus, the present invention not only automates human tasks but also realizes essential improvements in computer technology, such as AI-based recognition of data categories and optimization of advice algorithms.

The generation unit can estimate the user's emotion and adjust the length of advice based on the estimated emotion. For example, if the user is feeling stressed, the generation unit provides short and concise advice. The generation unit uses emotion estimation functions, such as emotion engines or generation AI, to estimate the user's emotion. For instance, the generation unit may use facial expression recognition technology to estimate the user's emotion by analyzing facial expression data captured by a camera. The generation unit can also use voice analysis technology to estimate the user's emotion by analyzing the tone and speed of the user's voice. Furthermore, if the user is relaxed, the generation unit provides detailed advice to facilitate deep understanding. For example, if the user is in a hurry, the generation unit provides concise advice. By adjusting the length of advice based on the user's emotion, advice of appropriate length can be provided to the user. Some or all of the above-described processes in the generation unit may be performed using AI or without using AI. For example, the generation unit may input the user's emotion data into a generation AI and have the generation AI adjust the length of advice. Specifically, the generation unit acquires multidimensional data such as facial expression feature vectors (e.g., 68 dimensions), voice features (e.g., MFCC, pitch, speech rate), and biosignals (e.g., heart rate, skin conductance activity) as time series tensors, applies noise removal and normalization (e.g., face image alignment, voice normalization, Z-score normalization of time series data), and inputs them into multimodal emotion recognition models based on CNN, RNN, or Transformer. Example inputs to the AI include (1) facial expression feature vectors per second (68 dimensions×30 frames), (2) 3 seconds of voice MFCC features (20 dimensions×30 frames), and (3) 30 seconds of heart rate time series (30 elements). From these inputs, the generation unit produces outputs such as (a) emotion labels (e.g., “stress,” “relaxation”), (b) emotion scores (e.g., 0-100), and (c) recommended advice length (e.g., “shortened,” “detailed,” “summary”). Example outputs include “emotion: stress,” “recommended length: shortened,” “emotion: relaxation,” “recommended length: detailed.” Based on these outputs, the advice generation module dynamically switches the length and detail of advice, providing “short sentences with key points” during stress, “detailed explanations” during relaxation, and “bullet-point summaries” when the user is in a hurry, optimizing the length of advice according to the user's psychological state and situation. The generation unit uses cross-entropy loss or mean squared error inside the AI model to continuously improve emotion estimation and expression optimization accuracy, applying transfer learning or online learning using user feedback and past emotion/understanding history. Unlike conventional subjective human judgment or fixed advice display, the generation unit achieves technical effects of optimal advice length in response to the user's psychological state and situation through high-dimensional multimodal data analysis and real-time control by AI. Application fields include health management for general consumers, stress monitoring, psychological state management for athletes, and mental health support in medical institutions. Thus, the present invention not only automates human tasks but also realizes essential improvements in computer technology, such as AI-based emotion recognition and optimization of advice length.

The generation unit can determine the priority of advice based on changes in health condition when generating advice. For example, if the heart rate changes rapidly, the generation unit prioritizes advice related to heart rate. The generation unit can use AI to evaluate changes in health condition. For instance, the generation unit may use machine learning algorithms to analyze changes in data and determine priorities. Additionally, if the stress level rises sharply, the generation unit can prioritize advice related to stress management. For example, the generation unit analyzes fluctuations in stress level and provides advice on stress management. Furthermore, if exercise data changes rapidly, the generation unit can prioritize advice related to exercise. For example, the generation unit analyzes fluctuations in exercise data and provides exercise advice. By determining the priority of advice based on changes in health condition, important health conditions can be prioritized for advice. Some or all of the above-described processes in the generation unit may be performed using AI or without using AI. For example, the generation unit may input health condition data into a generation AI and have the generation AI determine the priority of advice. Specifically, the generation unit acquires multidimensional health data such as heart rate time series data (e.g., 30-element vector per second), stress level scores (e.g., continuous values from 0 to 100), exercise data (e.g., acceleration vectors, calories burned), and blood pressure data (e.g., continuous values over 10 minutes) as time series tensors, applies noise removal and normalization (e.g., Z-score normalization, outlier removal), and inputs them into time series analysis models based on CNN, RNN, or Transformer. Example inputs to the AI include (1) 30 seconds of heart rate time series (30 elements), (2) 1 minute of stress level scores (60 elements), (3) exercise intensity vectors (10 elements), and (4) blood pressure continuous values (60 elements). From these inputs, the generation unit produces outputs such as (a) health condition change scores (e.g., continuous values from 0 to 1), (b) prioritized advice list (e.g., “heart rate advice>stress management advice>exercise advice”), and (c) recommended advice content (e.g., “deep breathing recommended for rapid heart rate increase,” “meditation recommended for rapid stress increase”). Example outputs include “change score: 0.85,” “priority advice: heart rate,” “recommended content: deep breathing.” Based on these outputs, the advice priority control module dynamically switches the order and content of advice generation for each data type, prioritizing advice for health indicators with rapid changes and providing immediate feedback to the user. The generation unit uses mean squared error or cross-entropy loss as the loss function inside the AI model and updates weights via gradient descent to continuously improve the accuracy of change detection and priority estimation. For individual optimization per user, the generation unit applies transfer learning or online learning, sequentially reflecting the user's past data and feedback in the learning process. The generation unit achieves technical effects of optimizing advice priority in response to individual changes in health condition, which was difficult with conventional human heuristics or uniform advice order, through high-dimensional time series analysis and automatic control by AI. Application fields include health management for general consumers, monitoring of chronic disease patients, recovery management for athletes, and remote monitoring in medical institutions. Thus, the present invention not only automates human tasks but also realizes essential improvements in computer technology, such as AI-based recognition of changes in health condition and optimization of advice priority.

The generation unit can adjust the order of advice based on the relevance of health conditions when generating advice. For example, the generation unit evaluates the relevance between heart rate data and stress level data and adjusts the order of advice based on highly relevant data. The generation unit can use AI to evaluate the relevance of health conditions. For instance, the generation unit may use machine learning algorithms to analyze the relevance of data and adjust the order of advice. Additionally, the generation unit evaluates the relevance between exercise data and calorie consumption data and adjusts the order of advice based on highly relevant data. For example, the generation unit analyzes the correlation between exercise data and calorie consumption data and adjusts the order of advice. Furthermore, the generation unit evaluates the relevance between meal data and nutritional balance data and adjusts the order of advice based on highly relevant data. For example, the generation unit analyzes the relevance between meal data and nutritional balance data and adjusts the order of advice. By adjusting the order of advice based on the relevance of health conditions, highly relevant advice can be prioritized. Some or all of the above-described processes in the generation unit may be performed using AI or without using AI. For example, the generation unit may input health condition data into a generation AI and have the generation AI adjust the order of advice. Specifically, the generation unit acquires multidimensional health data such as heart rate time series data (e.g., 30-element vector per second), stress level scores (e.g., continuous values from 0 to 100), exercise data (e.g., acceleration vectors, calories burned), meal record texts (e.g., “Lunch: salad, chicken, rice”), and nutritional balance data (e.g., intake amount vectors for each nutrient) as time series tensors or text, applies noise removal and normalization (e.g., Z-score normalization, outlier removal, text preprocessing), and inputs them into time series analysis models based on CNN, RNN, Transformer, or natural language processing models. Example inputs to the AI include (1) 30 seconds of heart rate time series (30 elements), (2) 1 minute of stress level scores (60 elements), (3) exercise intensity vectors (10 elements), (4) calorie consumption time series (10 elements), (5) meal record texts, and (6) nutrient intake amount vectors (10 elements). From these inputs, the generation unit produces outputs such as (a) data relevance scores (e.g., continuous values from 0 to 1), (b) advice order list (e.g., “heart rate→stress→exercise→calorie consumption”), and (c) recommended advice pairs (e.g., “exercise & calorie consumption,” “meal & nutritional balance”). Example outputs include “relevance: 0.85 (heart rate & stress),” “priority advice: heart rate & stress,” “relevance: 0.92 (exercise & calorie consumption).” Based on these outputs, the advice order control module dynamically switches the order and pairing of advice for each data type, prioritizing advice based on highly relevant health indicators and realizing comprehensive health condition evaluation and feedback. The generation unit uses mean squared error or cross-entropy loss as the loss function inside the AI model and updates weights via gradient descent to continuously improve the accuracy of relevance estimation and advice order optimization. For individual optimization per user, the generation unit applies transfer learning or online learning, sequentially reflecting the user's past data and feedback in the learning process. The generation unit achieves technical effects of optimizing advice order in response to data relevance, which was difficult with conventional human heuristics or uniform advice order, through high-dimensional data analysis and automatic control by AI. Application fields include health management for general consumers, monitoring of chronic disease patients, recovery management for athletes, and remote monitoring in medical institutions. Thus, the present invention not only automates human tasks but also realizes essential improvements in computer technology, such as AI-based recognition of data relevance and optimization of advice order.

The provision unit can estimate the user's emotion and adjust the method of expressing relaxation or mental health guidance based on the estimated emotion. For example, if the user is feeling stressed, the provision unit provides relaxation guidance in a calm voice. The provision unit uses emotion estimation functions, such as emotion engines or generation AI, to estimate the user's emotion. For instance, the provision unit may use facial expression recognition technology to estimate the user's emotion by analyzing facial expression data captured by a camera. The provision unit can also use voice analysis technology to estimate the user's emotion by analyzing the tone and speed of the user's voice. Furthermore, if the user is relaxed, the provision unit provides mental health guidance in a cheerful voice. For example, if the user is in a hurry, the provision unit provides quick and concise guidance. By adjusting the method of expressing relaxation or mental health guidance based on the user's emotion, guidance that is easy for the user to understand can be provided. Some or all of the above-described processes in the provision unit may be performed using AI or without using AI. For example, the provision unit may input the user's emotion data into a generation AI and have the generation AI adjust the method of expressing guidance. Specifically, the provision unit acquires multidimensional data such as facial expression feature vectors (e.g., 68 dimensions), voice features (e.g., MFCC, pitch, speech rate), and biosignals (e.g., heart rate, skin conductance activity) as time series tensors, applies noise removal and normalization (e.g., face image alignment, voice normalization, Z-score normalization of time series data), and inputs them into multimodal emotion recognition models based on CNN, RNN, or Transformer. Example inputs to the AI include (1) facial expression feature vectors per second (68 dimensions×30 frames), (2) 3 seconds of voice MFCC features (20 dimensions×30 frames), and (3) 30 seconds of heart rate time series (30 elements). From these inputs, the provision unit produces outputs such as (a) emotion labels (e.g., “stress,” “relaxation,” “anger”), (b) emotion scores (e.g., continuous values from 0 to 100), and (c) emotion trend (e.g., “stress increasing”). Example outputs include “emotion: stress,” “emotion score: 85,” “emotion trend: increasing.” Based on these outputs, the guide expression control module dynamically switches the guide's voice tone (e.g., calm voice, cheerful voice), speech rate (e.g., slow, fast), and expression granularity (e.g., detailed, concise), automatically generating the optimal guide expression according to the user's psychological state and situation. The provision unit uses cross-entropy loss or mean squared error inside the AI model to continuously improve emotion estimation and expression optimization accuracy, applying transfer learning or online learning using user feedback and past emotion/understanding history. Unlike conventional subjective human judgment or fixed guide expression, the provision unit achieves technical effects of optimal guide expression in response to the user's psychological state and situation through high-dimensional multimodal data analysis and real-time control by AI. Application fields include mental health support for general consumers, employee stress care in companies, rehabilitation support in medical institutions, and mental conditioning for athletes. Thus, the present invention not only automates human tasks but also realizes essential improvements in computer technology, such as AI-based emotion recognition and optimization of guide expression.

The provision unit can analyze the user's past stress levels when providing guidance and select the optimal guidance. For example, the provision unit analyzes the user's past stress level data and provides relaxation guidance during periods of high stress. The provision unit can use AI to analyze the user's past stress levels. For instance, the provision unit may use machine learning algorithms to analyze past data and select the optimal guidance. Additionally, the provision unit analyzes the user's past stress level data and provides mental health guidance during periods of low stress. For example, the provision unit predicts fluctuations in stress level based on past data and selects guidance. Furthermore, the provision unit analyzes the user's past stress level data and provides special guidance when stress rises sharply. For example, the provision unit analyzes sudden fluctuations in stress level and provides special guidance. By analyzing the user's past stress levels, the optimal guidance can be selected. Some or all of the above-described processes in the provision unit may be performed using AI or without using AI. For example, the provision unit may input past stress level data into a generation AI and have the generation AI select the optimal guidance. Specifically, the provision unit accumulates the user's stress level time series data for the past week to month (e.g., daily stress scores, hourly stress trend vectors) as time series tensors and inputs them into an AI analysis module. The provision unit first performs preprocessing such as missing value imputation, outlier removal, and time series normalization on the input data. For stress fluctuation prediction and anomaly detection, the provision unit uses autoencoders, LSTM-based time series prediction models, and time series clustering algorithms (e.g., k-means, DBSCAN). Example inputs to the AI include (1) 30 days of stress level time series (30 elements), (2) daily stress fluctuation vectors (30 elements), and (3) timestamps of sudden stress increase events (e.g., 3 events/week). From these inputs, the provision unit produces outputs such as (a) stress fluctuation prediction scores (e.g., continuous values from 0 to 1), (b) recommended guidance types (e.g., “relaxation guidance,” “mental health guidance,” “special guidance”), and (c) guidance provision timing (e.g., “high stress,” “low stress,” “sudden increase”). Example outputs include “stress fluctuation: high,” “recommended guidance: relaxation,” “provision timing: nighttime,” “sudden stress increase: yes,” “recommended guidance: special.” Based on these outputs, the guide selection module automatically selects the optimal guidance type and content for each time period or event and provides guidance to the user immediately or as scheduled. The provision unit uses reconstruction error or cross-entropy loss as the loss function inside the AI model and updates weights via gradient descent to continuously improve the accuracy of stress fluctuation prediction and guide selection. For individual optimization per user, the provision unit applies transfer learning or online learning, sequentially reflecting the user's past data and feedback in the learning process. The provision unit achieves technical effects of optimizing guide selection in response to individual stress states and life rhythms, which was difficult with conventional human heuristics or fixed guide provision, through high-dimensional time series analysis and automatic control by AI. Application fields include stress care for general consumers, employee mental health support in companies, stress monitoring in medical institutions, and psychological state management for athletes. Thus, the present invention not only automates human tasks but also realizes essential improvements in computer technology, such as AI-based recognition of stress fluctuations and optimization of guide selection.

The provision unit can customize the means of guidance based on the user's current living situation when providing guidance. For example, if the user is at work, the provision unit provides relaxation guidance that can be performed in a short time. The provision unit can use AI to evaluate the user's current living situation. For instance, the provision unit may use machine learning algorithms to analyze the living situation and customize the means of guidance. Additionally, if the user is at home, the provision unit can provide long-duration mental health guidance. For example, the provision unit analyzes the user's living situation and provides long-duration guidance. Furthermore, if the user is out, the provision unit can provide guidance that can be easily performed. For example, the provision unit analyzes the user's living situation and provides easily executable guidance. By customizing the means of guidance based on the user's current living situation, guidance that is easy for the user to implement can be provided. Some or all of the above-described processes in the provision unit may be performed using AI or without using AI. For example, the provision unit may input the user's living situation data into a generation AI and have the generation AI customize the means of guidance. Specifically, the provision unit acquires multidimensional data such as acceleration sensors, gyro sensors, GPS, calendar information, smartphone usage history, and environmental sensors (e.g., temperature, noise level) as time series tensors for estimating the user's living situation, applies noise removal and normalization (e.g., low-pass filtering of acceleration, Z-score normalization of environmental data), and inputs them into time series classification models based on CNN, RNN, or Transformer. Example inputs to the AI include (1) 10 seconds of three-axis acceleration data (3 dimensions×100 samples), (2) GPS location information (latitude, longitude, altitude as continuous values), (3) calendar events (e.g., meeting schedule, home schedule), (4) smartphone usage history (e.g., number of app launches, number of screen ON/OFF), and (5) environmental sound spectrum (e.g., FFT feature vector per second). From these inputs, the provision unit produces outputs such as (a) living situation labels (e.g., “at work,” “at home,” “out”), (b) recommended guidance means (e.g., “short-time relaxation,” “long-duration mental health,” “simple guidance”), and (c) recommended timing for guidance implementation (e.g., “lunch break,” “after returning home,” “while commuting”). Example outputs include “living situation: at work,” “recommended guidance: short-time relaxation,” “timing: lunch break.” Based on these outputs, the guide means customization module automatically selects the optimal guidance type, content, and implementation timing for each living situation and provides guidance to the user immediately or as scheduled. The provision unit uses cross-entropy loss or mean squared error inside the AI model to continuously improve the accuracy of living situation estimation and guide customization, applying transfer learning or online learning using user feedback and past living history. Unlike conventional subjective human judgment or fixed guide provision, the provision unit achieves technical effects of optimal guide means in response to individual living situations and behavior patterns through multidimensional data analysis and automatic control by AI. Application fields include health management for general consumers, employee stress care in companies, support for lifestyle-related diseases in medical institutions, and conditioning for athletes. Thus, the present invention not only automates human tasks but also realizes essential improvements in computer technology, such as AI-based recognition of living situations and optimization of guide means.

The provision unit can estimate the user's emotion and determine the priority of guidance based on the estimated emotion. For example, if the user is feeling stressed, the provision unit prioritizes relaxation guidance. The provision unit uses emotion estimation functions, such as emotion engines or generation AI, to estimate the user's emotion. For instance, the provision unit may use facial expression recognition technology to estimate the user's emotion by analyzing facial expression data captured by a camera. The provision unit can also use voice analysis technology to estimate the user's emotion by analyzing the tone and speed of the user's voice. Furthermore, if the user is relaxed, the provision unit prioritizes mental health guidance. For example, if the user is in a hurry, the provision unit prioritizes quick and concise guidance. By determining the priority of guidance based on the user's emotion, guidance necessary for the user can be prioritized. Some or all of the above-described processes in the provision unit may be performed using AI or without using AI. For example, the provision unit may input the user's emotion data into a generation AI and have the generation AI determine the priority of guidance. Specifically, the provision unit acquires multidimensional data such as facial expression feature vectors (e.g., 68 dimensions), voice features (e.g., MFCC, pitch, speech rate), and biosignals (e.g., heart rate, skin conductance activity) as time series tensors for emotion estimation, applies noise removal and normalization, and inputs them into multimodal emotion recognition models based on CNN, RNN, or Transformer. Example inputs to the AI include (1) facial expression feature vectors per second (68 dimensions×30 frames), (2) 3 seconds of voice MFCC features (20 dimensions×30 frames), and (3) 30 seconds of heart rate time series (30 elements). From these inputs, the provision unit produces outputs such as (a) emotion labels (e.g., “stress,” “relaxation”), (b) emotion scores (e.g., 0-100), and (c) recommended guide priority list (e.g., “relaxation guide>mental health guide>simple guide”). Example outputs include “emotion: stress,” “priority guide: relaxation,” “emotion: relaxation,” “priority guide: mental health.” Based on these outputs, the guide priority control module dynamically switches the order and content of guidance provision for each guide type, focusing on relaxation guidance during stress, mental health guidance during relaxation, and simple guidance when the user is in a hurry. The provision unit uses cross-entropy loss or mean squared error inside the AI model to continuously improve emotion estimation and priority optimization accuracy, applying transfer learning or online learning using user feedback and past emotion history. Unlike conventional subjective human judgment or fixed guide order, the provision unit achieves technical effects of real-time and highly accurate emotion recognition and guide priority control through multidimensional data analysis and automatic control by AI. Application fields include health management for general consumers, stress monitoring, psychological state management for athletes, and mental health support in medical institutions. Thus, the present invention not only automates human tasks but also realizes essential improvements in computer technology, such as AI-based emotion recognition and optimization of guide priority.

The provision unit can consider the user's geographic location information when providing guidance and select the optimal guidance. For example, if the user is at a high altitude, the provision unit provides relaxation guidance that considers oxygen saturation. The provision unit can use AI to consider the user's geographic location information. For instance, the provision unit may obtain the user's location information using a GPS sensor and analyze the data to identify the location. Additionally, if the user is in an urban area, the provision unit can provide mental health guidance that considers environmental sounds. For example, the provision unit analyzes environmental sounds and evaluates noise levels. Furthermore, if the user is in a natural environment, the provision unit can provide guidance that incorporates natural sounds. For example, the provision unit analyzes natural sounds and evaluates relaxation effects. By considering the user's geographic location information, the optimal guidance can be selected. Some or all of the above-described processes in the provision unit may be performed using AI or without using AI. For example, the provision unit may input the user's location information data into a generation AI and have the generation AI select the optimal guidance. Specifically, the provision unit acquires multidimensional data such as latitude, longitude, and altitude data from GPS sensors, barometric sensors, environmental sensors (e.g., temperature, humidity, noise, air pollution level), and surrounding audio data as time series tensors, applies noise removal and normalization (e.g., smoothing of location data, Z-score normalization of environmental data), and uses machine learning algorithms (e.g., decision trees, random forests, Transformer-based time series classification models) or acoustic analysis models. Example inputs to the AI include (1) current latitude, longitude, and altitude (3 dimensions), (2) movement trajectory over the past hour (60 samples), (3) time series data of temperature, humidity, and barometric pressure (each 60 elements), (4) noise level and air pollution index (each 60 elements), and (5) natural sound spectrum (e.g., FFT feature vector per second). From these inputs, the provision unit produces outputs such as (a) current location category (e.g., “high altitude,” “urban area,” “natural environment”), (b) recommended guidance type (e.g., “high altitude relaxation,” “urban mental health,” “natural sound guide”), and (c) environmental risk assessment (e.g., “high altitude risk high,” “urban noise high”). Example outputs include “current location: high altitude,” “recommended guide: relaxation considering oxygen saturation,” “environmental risk: high.” Based on these outputs, the guide selection module automatically selects the optimal guidance type and content for each geographic situation and environmental condition and provides guidance to the user immediately or as scheduled. The provision unit uses cross-entropy loss or mean squared error inside the AI model to continuously improve location recognition, risk estimation, and guide selection accuracy, applying transfer learning or online learning using the user's movement history and changes in health condition. Unlike conventional subjective location recognition or fixed guide provision, the provision unit achieves technical effects of real-time and highly accurate geographic risk recognition and guide selection optimization through multidimensional data analysis and automatic control by AI. Application fields include health management for mountaineers and high-altitude residents, environmental risk monitoring for urban dwellers, health management for travelers, and remote mental health support in medical institutions. Thus, the present invention not only automates human tasks but also realizes essential improvements in computer technology, such as AI-based geographic location recognition and optimization of guide selection.

The provision unit can analyze the user's social media activity when providing guidance and propose means of guidance. For example, if the user posts about feeling stressed on social media, the provision unit proposes relaxation guidance. The provision unit can use AI to analyze the user's social media activity. For instance, the provision unit may use natural language processing technology to analyze the content of social media posts and estimate emotions. Additionally, if the user posts about exercise, the provision unit can propose post-exercise relaxation guidance. For example, the provision unit analyzes the content of exercise-related posts and identifies the type and intensity of exercise. Furthermore, if the user posts about meals, the provision unit can propose post-meal mental health guidance. For example, the provision unit analyzes the content of meal-related posts and identifies the type and amount of food. By analyzing the user's social media activity, relevant means of guidance can be proposed. Some or all of the above-described processes in the provision unit may be performed using AI or without using AI. For example, the provision unit may input social media post data into a generation AI and have the generation AI propose means of guidance. Specifically, the provision unit inputs the user's social media post data (e.g., text, images, posting time, hashtags) into natural language processing modules or image analysis modules. For text data, the provision unit performs morphological analysis, word embedding (Word2Vec, BERT, etc.), sentiment analysis (e.g., labeling as positive, negative, stress), and topic classification (e.g., exercise, meal, stress). For image data, the provision unit uses CNN-based image classification models to estimate the content of exercise or meals. Example inputs to the AI include (1) post text (e.g., “I'm tired from work today,” “Achieved 5 km run”), (2) post images (e.g., meal photos, exercise selfies), (3) posting time and frequency data (e.g., number of posts per day), and (4) hashtags (e.g., “# stress,” “# workout”). From these inputs, the provision unit produces outputs such as (a) emotion labels (e.g., “stress,” “sense of achievement”), (b) topic labels (e.g., “exercise,” “meal”), and (c) recommended means of guidance (e.g., “relaxation guide,” “post-exercise guide,” “post-meal mental health guide”). Example outputs include “emotion: stress,” “recommended guide: relaxation,” “topic: exercise,” “recommended guide: post-exercise relaxation.” Based on these outputs, the guide means proposal module automatically selects the optimal guidance type and content for each post and emotional state and proposes guidance to the user immediately or as scheduled. The provision unit uses cross-entropy loss or mean squared error inside the AI model to continuously improve emotion/topic estimation and guide proposal accuracy, applying transfer learning or online learning using user feedback and past posting history. Unlike conventional subjective interpretation of posts or fixed guide proposals, the provision unit achieves technical effects of real-time and highly accurate social media analysis and optimization of means of guidance through multidimensional data analysis and automatic control by AI. Application fields include health management for general consumers, stress monitoring, training management for athletes, and support for lifestyle-related diseases in medical institutions. Thus, the present invention not only automates human tasks but also realizes essential improvements in computer technology, such as AI-based social media analysis and optimization of means of guidance.

The system according to the embodiment is not limited to the examples described above and can be variously modified as follows. Specifically, the system can flexibly change the module configuration and data flow of the collection unit, analysis unit, generation unit, and provision unit according to the user's usage environment and purpose. The system can combine and use various AI model architectures, such as convolutional neural networks, recurrent neural networks, Transformers, autoencoders, decision trees, and random forests. The system can be configured by arbitrarily combining sensor devices such as heart rate sensors, skin conductance activity sensors, acceleration sensors, GPS, environmental sensors, voice input devices, and cameras. The system can also execute each process of health data collection, analysis, guide generation, and provision in a distributed manner on different hardware such as cloud servers, edge devices, smartphones, and wearable devices. For individual optimization per user, the system can apply learning methods such as transfer learning, online learning, reinforcement learning, and federated learning, sequentially reflecting user feedback and past data in the learning process. The system is applicable to a wide range of fields, including health management, stress care, conditioning for athletes, remote monitoring in medical institutions, and employee mental health support in companies. The system can also add security functions such as data anonymization, encryption, and access control. Thus, the present invention not only automates human tasks but also realizes essential improvements in computer technology, such as AI-based health data analysis, guide generation, and individual optimization, in various configurations and operational forms.

The collection unit can adjust the timing for collecting data based on the user's sleep pattern when collecting the user's health data. For example, when the user is in deep sleep, the collection unit refrains from collecting data and collects data during light sleep. Additionally, the collection unit can evaluate the quality of the user's sleep and provide advice to improve sleep. By adjusting the timing for collecting data based on the user's sleep pattern, more accurate health data can be collected. Specifically, the collection unit acquires multidimensional time series data in real time for estimating the user's sleep state, such as three-axis acceleration vectors, heart rate per second, and continuous values of skin conductance activity from acceleration sensors, heart rate sensors, and skin conductance activity sensors. The collection unit applies preprocessing such as noise removal (e.g., low-pass filtering), normalization (e.g., Z-score normalization), and feature extraction (e.g., movement amount from acceleration, heart rate variability index) to these data. The collection unit uses time series classification models based on convolutional neural networks, recurrent neural networks, or Transformers to estimate sleep stages (e.g., wakefulness, light sleep, deep sleep, REM sleep). Example inputs to the AI include (1) 10 minutes of three-axis acceleration data (3 dimensions×600 samples), (2) 10 minutes of heart rate time series (600 elements), and (3) continuous values of skin conductance activity (600 elements). From these inputs, the collection unit produces outputs such as (a) sleep stage labels (e.g., “deep sleep,” “light sleep”), (b) sleep quality scores (e.g., 0-100), and (c) recommended data collection timing (e.g., “refrain from collecting,” “collect”). Example outputs include “sleep stage: deep sleep,” “recommended collection: refrain,” “sleep quality: 85.” Based on these outputs, the data collection control module dynamically switches the sensor operation timing and sampling rate, minimizing sensor operation during deep sleep and focusing data collection during light sleep or wakefulness. The collection unit uses cross-entropy loss or mean squared error inside the AI model to continuously improve sleep stage estimation and collection timing optimization accuracy, applying transfer learning or online learning using user feedback and past sleep history. Unlike conventional subjective sleep records or fixed data collection schedules, the collection unit achieves technical effects of real-time and highly accurate sleep pattern recognition and data collection optimization through multidimensional time series analysis and automatic control by AI. Application fields include sleep management for general consumers, monitoring of sleep disorder patients, recovery management for athletes, and remote sleep evaluation in medical institutions. Thus, the present invention not only automates human tasks but also realizes essential improvements in computer technology, such as AI-based sleep pattern recognition and optimization of data collection timing.

The analysis unit can improve the accuracy of analysis by considering the user's past health history when analyzing the user's health data. For example, the analysis unit grasps trends in the user's health condition based on past data and enhances the accuracy of abnormal value detection. Additionally, the analysis unit can predict specific health risks by considering family history and genetic information. By considering the user's past health history, more accurate analysis results can be provided. Specifically, the analysis unit accumulates the user's health data for the past month to year (e.g., heart rate time series, blood pressure trends, stress level history, exercise history, meal records) as time series tensors or structured data and inputs them into an AI analysis module. The analysis unit performs preprocessing such as missing value imputation, outlier removal, time series normalization, and feature engineering (e.g., moving average, trend extraction, periodicity analysis) on the input data. The analysis unit uses LSTM or Transformer-based time series prediction models and anomaly detection algorithms (e.g., autoencoders, Isolation Forest) to estimate health condition trends and detect abnormal values. Example inputs to the AI include (1) daily average heart rate for the past 365 days (365 elements), (2) weekly blood pressure trend vectors (52 elements), (3) stress level time series (365 elements), and (4) family history/genetic information vectors (e.g., disease presence flags, genetic risk scores). From these inputs, the analysis unit produces outputs such as (a) health trend evaluation (e.g., “heart rate increasing trend,” “blood pressure stable”), (b) anomaly detection labels (e.g., “abnormal value: yes,” “abnormal value: no”), and (c) health risk prediction (e.g., “high risk of hypertension,” “medium risk of diabetes”). Example outputs include “trend: heart rate increasing,” “abnormal: yes,” “risk: hypertension.” Based on these outputs, the analysis result generation module automatically generates optimal analysis content and feedback for each user, performing detailed cause analysis and risk assessment when anomalies are detected. The analysis unit uses mean squared error or cross-entropy loss as the loss function inside the AI model and updates weights via gradient descent to continuously improve the accuracy of trend estimation, anomaly detection, and risk prediction. For individual optimization per user, the analysis unit applies transfer learning or online learning, sequentially reflecting the user's past data and feedback in the learning process. The analysis unit achieves technical effects of high-precision health condition analysis and risk prediction in response to individual health history and genetic background, which was difficult with conventional human heuristics or uniform analysis, through high-dimensional time series analysis and automatic control by AI. Application fields include health management for general consumers, risk monitoring for chronic disease patients, preventive medicine for hereditary diseases, and personalized diagnostic support in medical institutions. Thus, the present invention not only automates human tasks but also realizes essential improvements in computer technology, such as AI-based health history analysis and optimization of risk prediction.

The generation unit can provide customized advice by considering the user's lifestyle and preferences when generating advice based on the user's health condition. For example, if the user is a vegetarian, the generation unit provides nutrition advice for vegetarians. Additionally, if the user leads a busy life, the generation unit can provide exercise advice that can be performed in a short time. By providing advice tailored to the user's lifestyle and preferences, health management that is easy to implement can be supported. Specifically, the generation unit acquires the user's health condition data (e.g., heart rate, blood pressure, stress level, exercise history, meal records), lifestyle information (e.g., occupation, working hours, exercise habits, dietary preferences, allergy information, religious restrictions), and preference data (e.g., favorite exercise types, foods, cooking methods) as structured data. The generation unit performs preprocessing such as category encoding, one-hot vectorization, and numerical normalization on these data and inputs them into multi-input integration models based on Transformers or gradient boosting decision tree models. Example inputs to the AI include (1) health condition vector (e.g., heart rate 80, blood pressure 120/80, stress level 40), (2) lifestyle vector (e.g., vegetarian, exercise three times a week, work 8 hours a day), and (3) preference vector (e.g., likes Japanese food, allergic to dairy products). From these inputs, the generation unit produces outputs such as (a) recommended advice type (e.g., “nutrition advice for vegetarians,” “short-time exercise proposal”), (b) advice content (e.g., “actively consume beans and green-yellow vegetables,” “recommend 5-minute stretching”), and (c) recommended timing for implementation (e.g., “at breakfast,” “during lunch break,” “after returning home”). Example outputs include “advice: protein intake for vegetarians,” “content: recommend tofu and soybean products,” “timing: at dinner.” Based on these outputs, the advice generation module automatically generates optimal advice content and expression for each user and presents it in an easy-to-implement form. The generation unit uses cross-entropy loss or mean squared error inside the AI model to continuously improve advice content optimization accuracy, applying transfer learning or online learning using user feedback and implementation history. Unlike conventional uniform advice or subjective proposals by humans, the generation unit achieves technical effects of optimal advice in response to individual lifestyle and preferences through multidimensional data analysis and automatic control by AI. Application fields include health management for general consumers, nutrition guidance for people with specific dietary restrictions, health support for busy businesspersons, and personalized health guidance in medical institutions. Thus, the present invention not only automates human tasks but also realizes essential improvements in computer technology, such as AI-based recognition of lifestyle and preferences and optimization of advice.

The provision unit can customize the content of relaxation or mental health guidance based on the user's music preferences when providing guidance based on the user's health data. For example, if the user prefers classical music, the provision unit provides meditation guidance with classical music as background. Additionally, if the user prefers pop music, the provision unit can provide relaxation guidance incorporating pop music. By providing guidance tailored to the user's music preferences, the relaxation effect can be enhanced. Specifically, the provision unit acquires the user's music preference data (e.g., playback history by genre, playlists from music streaming services, preference information from surveys) as structured data and combines it with health condition data (e.g., stress level, heart rate, sleep quality) for input into an AI analysis module. The provision unit performs preprocessing such as genre classification, feature vectorization (e.g., one-hot encoding for classical, pop, jazz, rock), and normalization of playback frequency on music preference data. The provision unit uses multi-input integration models based on Transformers or gradient boosting decision tree models to estimate the optimal guidance content from the combination of health condition and music preference. Example inputs to the AI include (1) stress level score (0-100), (2) music genre preference vector (e.g., classical 0.8, pop 0.2), and (3) sleep quality score (0-100). From these inputs, the provision unit produces outputs such as (a) recommended guidance type (e.g., “classical music meditation guide,” “pop music relaxation guide”), (b) guidance content (e.g., “meditation with Bach's unaccompanied cello suites as BGM,” “breathing exercises using up-tempo pop songs”), and (c) recommended timing for implementation (e.g., “before bedtime,” “during lunch break”). Example outputs include “guide: classical music meditation,” “content: Bach BGM meditation,” “timing: nighttime.” Based on these outputs, the guide generation module automatically generates optimal music-accompanied guidance content and expression for each user, maximizing the relaxation effect. The provision unit uses cross-entropy loss or mean squared error inside the AI model to continuously improve guidance content optimization accuracy, applying transfer learning or online learning using user feedback and implementation history. Unlike conventional uniform guidance or subjective music selection by humans, the provision unit achieves technical effects of optimal guidance in response to individual music preferences through multidimensional data analysis and automatic control by AI. Application fields include mental health support for general consumers, stress care, sleep improvement, and relaxation guidance in medical institutions. Thus, the present invention not only automates human tasks but also realizes essential improvements in computer technology, such as AI-based recognition of music preferences and optimization of guidance.

The collection unit can estimate the user's emotion and adjust the method of collecting health data based on the estimated emotion. For example, if the user is feeling stressed, the collection unit prioritizes the collection of data related to stress level. Additionally, if the user is relaxed, the collection unit can collect data related to relaxation state. Furthermore, the collection unit can adjust the frequency of data collection based on the user's emotion and collect only the necessary data. By adjusting the method of collecting health data based on the user's emotion, more appropriate data can be collected. Specifically, the collection unit acquires multidimensional data such as facial expression feature vectors (e.g., 68 dimensions), voice features (e.g., MFCC, pitch, speech rate), and biosignals (e.g., heart rate, skin conductance activity) as time series tensors for emotion estimation, applies noise removal and normalization (e.g., face image alignment, voice normalization, Z-score normalization of time series data), and inputs them into multimodal emotion recognition models based on CNN, RNN, or Transformer. Example inputs to the AI include (1) facial expression feature vectors per second (68 dimensions×30 frames), (2) 3 seconds of voice MFCC features (20 dimensions×30 frames), and (3) 30 seconds of heart rate time series (30 elements). From these inputs, the collection unit produces outputs such as (a) emotion labels (e.g., “stress,” “relaxation”), (b) emotion scores (e.g., 0-100), (c) recommended data types for collection (e.g., “stress level,” “relaxation index”), and (d) recommended collection frequency (e.g., “high frequency,” “low frequency”). Example outputs include “emotion: stress,” “recommended collection: stress level, high frequency,” “emotion: relaxation,” “recommended collection: relaxation index, low frequency.” Based on these outputs, the collection control module dynamically switches the data acquisition targets and frequency for each sensor, focusing on stress-related data during stress posts and relaxation-related data during relaxation. The collection unit uses cross-entropy loss or mean squared error inside the AI model to continuously improve emotion estimation and collection optimization accuracy, applying transfer learning or online learning using user feedback and past emotion history. Unlike conventional subjective emotion judgment or fixed data collection, the collection unit achieves technical effects of real-time and highly accurate emotion recognition and data collection optimization through multidimensional data analysis and automatic control by AI. Application fields include health management for general consumers, stress monitoring, psychological state management for athletes, and mental health support in medical institutions. Thus, the present invention not only automates human tasks but also realizes essential improvements in computer technology, such as AI-based emotion recognition and optimization of data collection.

The analysis unit can estimate the user's emotion and determine the priority of analysis based on the estimated emotion. For example, if the user is feeling stressed, the analysis unit prioritizes the analysis of data related to stress level. Additionally, if the user is relaxed, the analysis unit can prioritize the analysis of data related to relaxation state. Furthermore, the analysis unit can adjust the level of detail of analysis based on the user's emotion and provide only the necessary information. By determining the priority of analysis based on the user's emotion, important data can be prioritized for analysis. Specifically, the analysis unit acquires multidimensional data such as facial expression feature vectors, voice features, and biosignals as time series tensors for emotion estimation, applies noise removal and normalization, and inputs them into multimodal emotion recognition models based on CNN, RNN, or Transformer. Example inputs to the AI include (1) facial expression feature vectors per second (68 dimensions×30 frames), (2) 3 seconds of voice MFCC features (20 dimensions×30 frames), and (3) 30 seconds of heart rate time series (30 elements). From these inputs, the analysis unit produces outputs such as (a) emotion labels (e.g., “stress,” “relaxation”), (b) emotion scores (e.g., 0-100), (c) recommended analysis priority list (e.g., “stress-related data>relaxation-related data”), and (d) recommended analysis detail level (e.g., “detailed,” “simple”). Example outputs include “emotion: stress,” “priority analysis: stress level, detailed,” “emotion: relaxation,” “priority analysis: relaxation index, simple.” Based on these outputs, the analysis priority control module dynamically switches the order and detail level of analysis for each data type, performing detailed analysis of stress-related data during stress and simple analysis of relaxation-related data during relaxation. The analysis unit uses cross-entropy loss or mean squared error inside the AI model to continuously improve emotion estimation and analysis optimization accuracy, applying transfer learning or online learning using user feedback and past emotion history. Unlike conventional subjective emotion judgment or fixed analysis order, the analysis unit achieves technical effects of real-time and highly accurate emotion recognition and optimization of analysis priority through multidimensional data analysis and automatic control by AI. Application fields include health management for general consumers, stress monitoring, psychological state management for athletes, and mental health support in medical institutions. Thus, the present invention not only automates human tasks but also realizes essential improvements in computer technology, such as AI-based emotion recognition and optimization of analysis priority.

The generation unit can estimate the user's emotion and adjust the content of advice based on the estimated emotion. For example, if the user is feeling stressed, the generation unit provides advice focused on relaxation. Additionally, if the user is relaxed, the generation unit can provide advice on maintaining health. Furthermore, the generation unit can adjust the tone and method of expressing advice based on the user's emotion to provide advice that is easy for the user to understand. By adjusting the content of advice based on the user's emotion, more effective health management can be supported. Specifically, the generation unit acquires multidimensional data such as facial expression feature vectors, voice features, and biosignals as time series tensors for emotion estimation, applies noise removal and normalization, and inputs them into multimodal emotion recognition models based on CNN, RNN, or Transformer. Example inputs to the AI include (1) facial expression feature vectors per second (68 dimensions×30 frames), (2) 3 seconds of voice MFCC features (20 dimensions×30 frames), and (3) 30 seconds of heart rate time series (30 elements). From these inputs, the generation unit produces outputs such as (a) emotion labels (e.g., “stress,” “relaxation”), (b) emotion scores (e.g., 0-100), (c) recommended advice content (e.g., “focus on relaxation,” “focus on health maintenance”), and (d) recommended advice tone (e.g., “calm,” “cheerful”). Example outputs include “emotion: stress,” “advice content: relaxation,” “tone: calm,” “emotion: relaxation,” “advice content: health maintenance,” “tone: cheerful.” Based on these outputs, the advice generation module dynamically switches the content and method of expressing advice, presenting relaxation-focused advice in a calm tone during stress and health maintenance-focused advice in a cheerful tone during relaxation. The generation unit uses cross-entropy loss or mean squared error inside the AI model to continuously improve emotion estimation and advice optimization accuracy, applying transfer learning or online learning using user feedback and past emotion/understanding history. Unlike conventional subjective emotion judgment or fixed advice content, the generation unit achieves technical effects of real-time and highly accurate emotion recognition and optimization of advice content through multidimensional data analysis and automatic control by AI. Application fields include health management for general consumers, stress monitoring, psychological state management for athletes, and mental health support in medical institutions. Thus, the present invention not only automates human tasks but also realizes essential improvements in computer technology, such as AI-based emotion recognition and optimization of advice content.

The provision unit can estimate the user's emotion and adjust the content of relaxation or mental health guidance based on the estimated emotion. For example, if the user is feeling stressed, the provision unit provides guidance effective for stress reduction. Additionally, if the user is relaxed, the provision unit can provide guidance for maintaining the relaxation state. Furthermore, the provision unit can adjust the length and level of detail of guidance based on the user's emotion to provide optimal guidance for the user. By adjusting the content of guidance based on the user's emotion, more effective support for relaxation and mental health can be provided. Specifically, the provision unit acquires multidimensional data such as facial expression feature vectors, voice features, and biosignals as time series tensors for emotion estimation, applies noise removal and normalization, and inputs them into multimodal emotion recognition models based on CNN, RNN, or Transformer. Example inputs to the AI include (1) facial expression feature vectors per second (68 dimensions×30 frames), (2) 3 seconds of voice MFCC features (20 dimensions×30 frames), and (3) 30 seconds of heart rate time series (30 elements). From these inputs, the provision unit produces outputs such as (a) emotion labels (e.g., “stress,” “relaxation”), (b) emotion scores (e.g., 0-100), (c) recommended guidance content (e.g., “stress reduction,” “relaxation maintenance”), and (d) recommended guidance length/detail level (e.g., “detailed,” “simple”). Example outputs include “emotion: stress,” “guidance content: stress reduction,” “length: detailed,” “emotion: relaxation,” “guidance content: relaxation maintenance,” “length: simple.” Based on these outputs, the guide generation module dynamically switches the content, length, and level of detail of guidance, presenting detailed guidance focused on stress reduction during stress and simple guidance focused on relaxation maintenance during relaxation. The provision unit uses cross-entropy loss or mean squared error inside the AI model to continuously improve emotion estimation and guidance optimization accuracy, applying transfer learning or online learning using user feedback and past emotion/understanding history. Unlike conventional subjective emotion judgment or fixed guidance content, the provision unit achieves technical effects of real-time and highly accurate emotion recognition and optimization of guidance content through multidimensional data analysis and automatic control by AI. Application fields include health management for general consumers, stress monitoring, psychological state management for athletes, and mental health support in medical institutions. Thus, the present invention not only automates human tasks but also realizes essential improvements in computer technology, such as AI-based emotion recognition and optimization of guidance content.

The provision unit is capable of estimating a user's emotion and adjusting the method of providing guidance based on the estimated emotion of the user. For example, when the user is feeling stressed, relaxation guidance is provided in a calm voice. Additionally, when the user is relaxed, mental health guidance may be provided in a cheerful voice. Furthermore, the provision unit can adjust the timing of guidance provision based on the user's emotion, thereby providing guidance at the optimal timing for the user. By adjusting the method of guidance provision based on the user's emotion, more effective support for relaxation and mental health can be achieved. Specifically, the provision unit acquires multidimensional data such as facial expression feature vectors, voice features, and biosignals as time-series tensors for emotion estimation, applies noise reduction and normalization, and inputs the processed data into convolutional neural networks, recurrent neural networks, or Transformer-based multimodal emotion recognition models. As examples of input to the AI, the provision unit uses (1) facial expression feature vectors per second (68 dimensions×30 frames), (2) 3-second voice MFCC features (20 dimensions×30 frames), and (3) 30-second heart rate time series (30 elements). From these inputs, the provision unit generates outputs such as (a) emotion labels (e.g., “stress”, “relaxation”), (b) emotion scores (e.g., 0-100), (c) recommended guidance provision methods (e.g., “calm voice”, “cheerful voice”), and (d) recommended guidance provision timing (e.g., “immediate”, “before bedtime”, “morning”). Examples of output include “Emotion: Stress”, “Provision Method: Calm Voice”, “Timing: Immediate”; “Emotion: Relaxation”, “Provision Method: Cheerful Voice”, “Timing: Morning”. Based on these outputs, the guidance provision control module dynamically switches the voice tone and timing of guidance provision, presenting immediate guidance in a calm voice during stress and morning guidance in a cheerful voice during relaxation. The provision unit continuously improves emotion estimation accuracy and guidance provision optimization accuracy within the AI model by using cross-entropy loss and mean squared error, and applies transfer learning and online learning using user feedback and historical emotion/understanding records. The provision unit achieves real-time and highly accurate emotion recognition and guidance provision method optimization, which were difficult with conventional subjective human emotion judgment and fixed guidance provision methods, through AI-based multidimensional data analysis and automatic control. Application fields include health management for general consumers, stress monitoring, psychological state management for athletes, and mental health support in medical institutions. Thus, the present invention not only automates human tasks but also realizes an essential improvement in computer technology through AI-based emotion recognition and guidance provision method optimization.

The provision unit is capable of estimating a user's emotion and determining the priority of guidance based on the estimated emotion of the user. For example, when the user is feeling stressed, relaxation guidance is preferentially provided. Additionally, when the user is relaxed, mental health guidance may be preferentially provided. Furthermore, the provision unit can adjust the content of guidance based on the user's emotion, thereby providing optimal guidance for the user. By determining the priority of guidance based on the user's emotion, guidance necessary for the user can be preferentially provided. Specifically, the provision unit acquires multidimensional data such as facial expression feature vectors, voice features, and biosignals as time-series tensors for emotion estimation, applies noise reduction and normalization, and inputs the processed data into convolutional neural networks, recurrent neural networks, or Transformer-based multimodal emotion recognition models. As examples of input to the AI, the provision unit uses (1) facial expression feature vectors per second (68 dimensions×30 frames), (2) 3-second voice MFCC features (20 dimensions×30 frames), and (3) 30-second heart rate time series (30 elements). From these inputs, the provision unit generates outputs such as (a) emotion labels (e.g., “stress”, “relaxation”), (b) emotion scores (e.g., 0-100), (c) recommended guidance priority lists (e.g., “relaxation guidance>mental health guidance>simple guidance”), and (d) recommended guidance content (e.g., “stress reduction”, “maintenance of relaxation”). Examples of output include “Emotion: Stress”, “Priority Guidance: Relaxation”, “Content: Stress Reduction”; “Emotion: Relaxation”, “Priority Guidance: Mental Health”, “Content: Maintenance of Relaxation”. Based on these outputs, the guidance priority control module dynamically switches the order and content of guidance for each guidance type, focusing on relaxation guidance during stress and mental health guidance during relaxation. The provision unit continuously improves emotion estimation accuracy and priority optimization accuracy within the AI model by using cross-entropy loss and mean squared error, and applies transfer learning and online learning using user feedback and historical emotion records. The provision unit achieves real-time and highly accurate emotion recognition and guidance priority control, which were difficult with conventional subjective human emotion judgment and fixed guidance order, through AI-based multidimensional data analysis and automatic control. Application fields include health management for general consumers, stress monitoring, psychological state management for athletes, and mental health support in medical institutions. Thus, the present invention not only automates human tasks but also realizes an essential improvement in computer technology through AI-based emotion recognition and guidance priority optimization.

The following is a brief description of the processing flow of Example of the Embodiment. Specifically, the present system operates in cooperation among the collection unit, analysis unit, generation unit, and provision unit, executing a series of data flows from acquisition of the user's health data, analysis, advice generation, to guidance provision. The system enables the collection unit to acquire various health-related data in real time, such as heart rate, blood pressure, stress level, sleep data, exercise data, dietary records, and voice/image data, and accumulates these as time-series tensors or structured data. The analysis unit performs noise reduction, normalization, and feature extraction on the collected data, and uses convolutional neural networks, recurrent neural networks, Transformer-based time-series analysis models, and natural language processing models to conduct health condition evaluation, anomaly detection, emotion estimation, data category classification, and trend analysis. The generation unit generates individually optimized advice and guidance content by considering the analysis results and the user's lifestyle, preferences, and emotional state. The provision unit presents the generated advice and guidance in the optimal expression method, timing, and priority according to the user's emotion, situation, and preferences. Each module continuously performs learning and optimization within the AI model using loss functions (such as cross-entropy loss and mean squared error), and achieves individual optimization through transfer learning and online learning using user feedback and past data. Thus, the present invention achieves the automation and optimization of real-time and highly accurate health condition analysis, advice generation, and guidance provision, which were difficult with conventional subjective and uniform health management by humans, through AI-based multidimensional data analysis and automatic control. Application fields include health management for general consumers, monitoring of chronic disease patients, conditioning for athletes, and remote health support in medical institutions. Thus, the present invention not only automates human tasks but also realizes an essential improvement in computer technology through AI-based health data analysis, advice generation, and guidance provision optimization.

Step 1: The collection unit collects health data of the user. The user's health data includes heart rate, blood pressure, stress level, and the like. The collection unit collects the user's health data in real time using a heart rate sensor or a sensor configured to measure a stress level. The heart rate sensor measures heart rate using an optical sensor or an electrical sensor, and the sensor configured to measure a stress level measures stress level using a skin electrical activity sensor or a heart rate variability sensor. Step 2: The analysis unit analyzes the health data collected by the collection unit. The analysis is performed using statistical analysis or machine learning algorithms, analyzing the collected health data and evaluating the user's health condition. The health condition evaluation includes calculation of a health score and detection of abnormal values. Step 3: The generation unit generates advice based on the analysis result obtained by the analysis unit. The advice includes exercise proposals and dietary recommendations, and appropriate exercise or nutrition advice is generated based on the user's health condition. Step 4: The provision unit provides relaxation or mental health guidance based on the advice generated by the generation unit. The relaxation or mental health guidance includes meditation instruction and stress management techniques, and is provided based on the user's stress level. Specifically, in Step 1, the system enables the collection unit to acquire multidimensional data in real time, such as heart rate per second using a heart rate sensor (e.g., optical PPG sensor, electrical ECG sensor), blood pressure per 10 minutes using a blood pressure sensor (e.g., cuff-type, cuffless-type), skin electrical activity sensor, accelerometer, voice input device, camera, and the like, as well as stress level per minute, exercise intensity vector, facial expression feature vector, and voice features. The collection unit performs preprocessing such as noise reduction (e.g., low-pass filter), normalization (e.g., Z-score normalization), and feature extraction (e.g., heart rate variability index, movement amount from acceleration, voice MFCC) on these data. In Step 2, the analysis unit uses convolutional neural networks, recurrent neural networks, Transformer-based time-series analysis models, and natural language processing models to perform health condition evaluation (e.g., health score 0-100), anomaly detection (e.g., “heart rate anomaly: present”), emotion estimation (e.g., “stress”, “relaxation”), data category classification (e.g., “heart rate”, “stress”), and trend analysis (e.g., “increasing trend in heart rate”). Examples of input to the AI include (1) 30-second heart rate time series (30 elements), (2) 1-minute stress level score (60 elements), (3) exercise intensity vector (10 elements), (4) facial expression feature vector (68 dimensions×30 frames), and (5) voice MFCC features (20 dimensions×30 frames). The analysis unit generates outputs such as health score, anomaly detection label, emotion label, and trend evaluation from these inputs and passes them to the analysis result generation module. In Step 3, the generation unit generates individually optimized advice (e.g., “5-minute stretch recommended”, “tofu/soy products recommended”) and guidance content (e.g., “Bach BGM meditation”, “breathing technique using up-tempo pop music”) by considering the analysis results and the user's lifestyle, preferences, and emotional state. The generation unit continuously improves advice content optimization accuracy within the AI model by using cross-entropy loss and mean squared error, and applies transfer learning and online learning using user feedback and implementation history. In Step 4, the provision unit presents the generated advice and guidance in the optimal expression method, timing, and priority according to the user's emotion, situation, and preferences. The provision unit dynamically switches guidance content, voice tone, length, level of detail, and provision timing to realize optimal support for relaxation and mental health for the user. Thus, the present invention achieves the automation and optimization of real-time and highly accurate health condition analysis, advice generation, and guidance provision, which were difficult with conventional subjective and uniform health management by humans, through AI-based multidimensional data analysis and automatic control. Application fields include health management for general consumers, monitoring of chronic disease patients, conditioning for athletes, and remote health support in medical institutions. Thus, the present invention not only automates human tasks but also realizes an essential improvement in computer technology through AI-based health data analysis, advice generation, and guidance provision optimization.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of the plurality of elements including the above-described collection unit, analysis unit, generation unit, and provision unit is implemented by at least one of, for example, a smart device 14 and a data processing apparatus 12. For example, the collection unit collects health data of a user in real time using a heart rate sensor or a sensor configured to measure a stress level of the smart device 14. The analysis unit is implemented, for example, by a specific processing unit 290 of the data processing apparatus 12 and analyzes the collected health data. The generation unit is implemented, for example, by the specific processing unit 290 of the data processing apparatus 12 and generates advice based on the analysis result. The provision unit is implemented, for example, by a control unit 46A of the smart device 14 and provides relaxation or mental health guidance based on the generated advice. The correspondence between each unit and the apparatus or control unit is not limited to the above examples and various modifications are possible.

### Second Embodiment

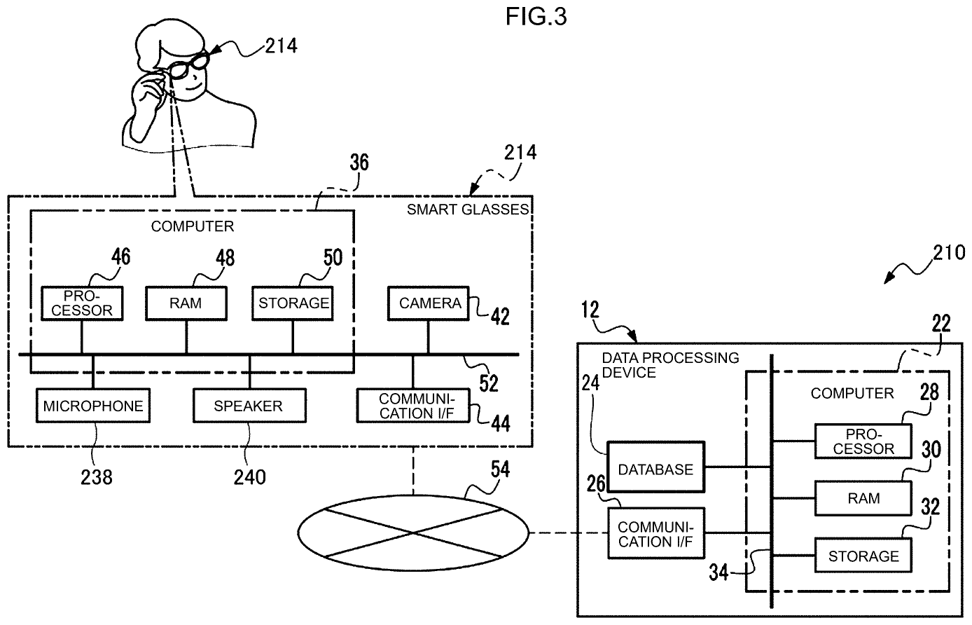

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

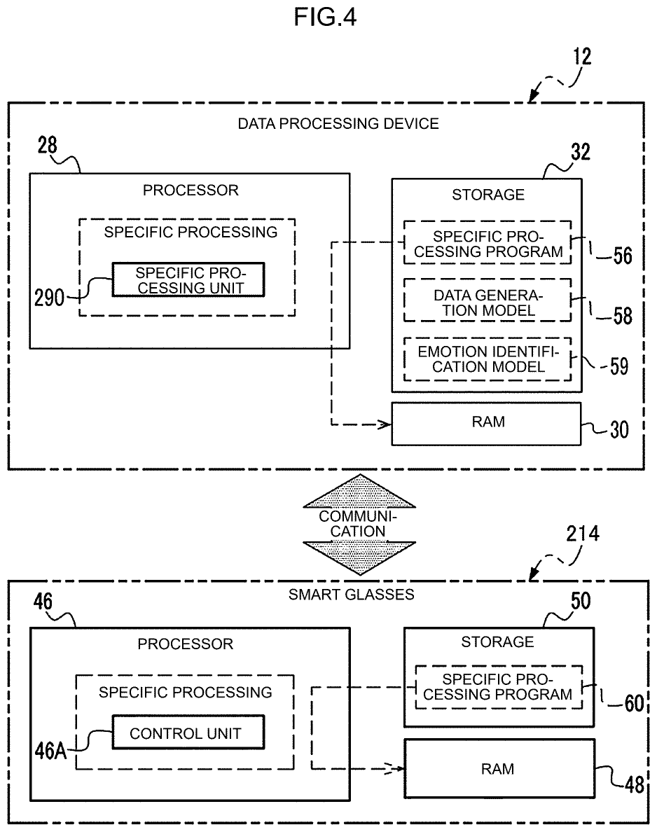

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of the plurality of elements including the above-described collection unit, analysis unit, generation unit, and provision unit is implemented by at least one of, for example, smart glasses 214 and a data processing apparatus 12. For example, the collection unit collects health data of a user in real time using a heart rate sensor or a sensor configured to measure a stress level of the smart glasses 214. The analysis unit is implemented, for example, by a specific processing unit 290 of the data processing apparatus 12 and analyzes the collected health data. The generation unit is implemented, for example, by the specific processing unit 290 of the data processing apparatus 12 and generates advice based on the analysis result. The provision unit is implemented, for example, by a control unit 46A of the smart glasses 214 and provides relaxation or mental health guidance based on the generated advice. The correspondence between each unit and the apparatus or control unit is not limited to the above examples and various modifications are possible.

### Third Embodiment

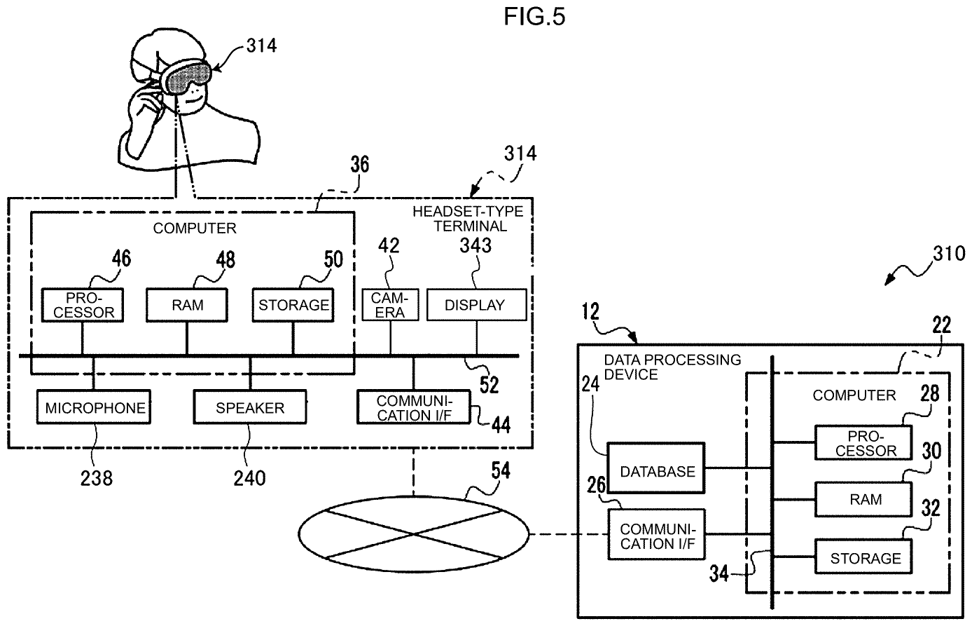

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

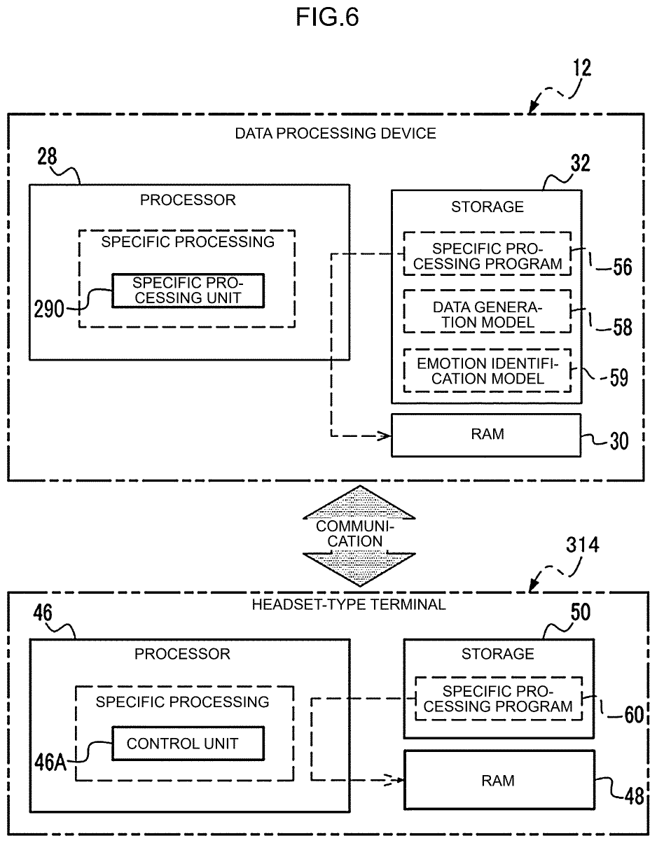

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of the plurality of elements including the above-described collection unit, analysis unit, generation unit, and provision unit is implemented by at least one of, for example, a headset-type terminal 314 and a data processing apparatus 12. For example, the collection unit collects health data of a user in real time using a heart rate sensor or a sensor configured to measure a stress level of the headset-type terminal 314. The analysis unit is implemented, for example, by a specific processing unit 290 of the data processing apparatus 12 and analyzes the collected health data. The generation unit is implemented, for example, by the specific processing unit 290 of the data processing apparatus 12 and generates advice based on the analysis result. The provision unit is implemented, for example, by a control unit 46A of the headset-type terminal 314 and provides relaxation or mental health guidance based on the generated advice. The correspondence between each unit and the apparatus or control unit is not limited to the above examples and various modifications are possible.

### Fourth Embodiment

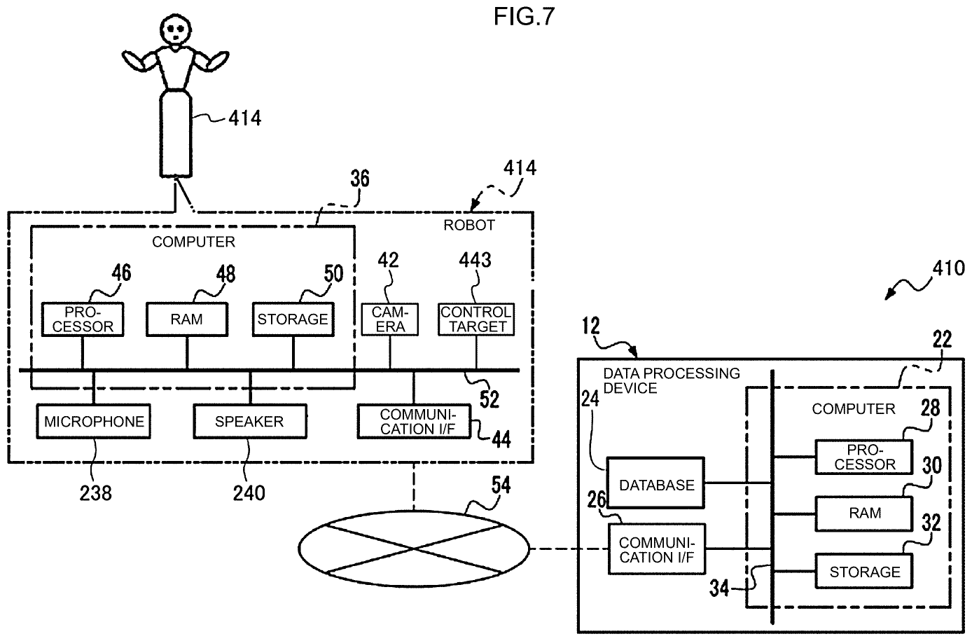

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

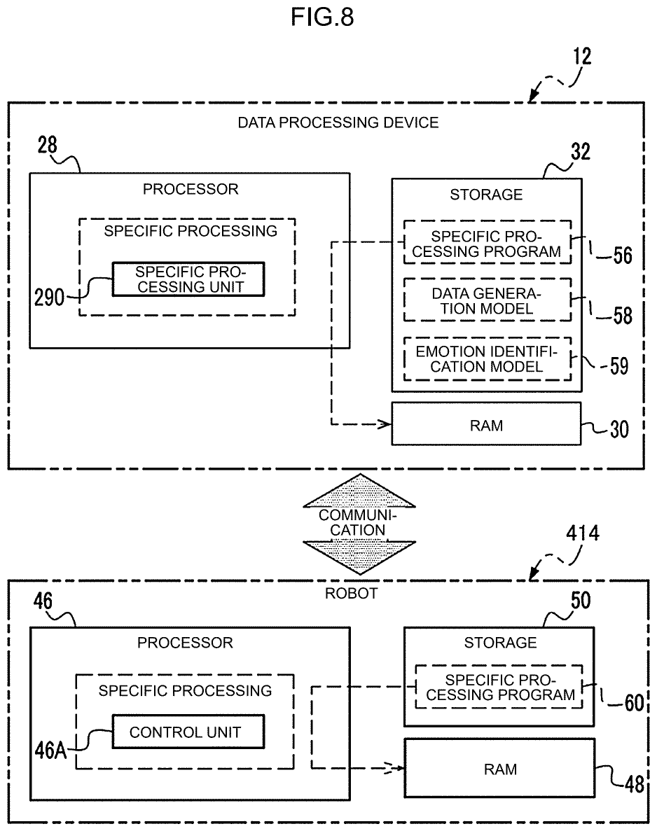

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of the plurality of elements including the above-described collection unit, analysis unit, generation unit, and provision unit is implemented by at least one of, for example, a robot 414 and a data processing apparatus 12. For example, the collection unit collects health data of a user in real time using a heart rate sensor or a sensor configured to measure a stress level of the robot 414. The analysis unit is implemented, for example, by a specific processing unit 290 of the data processing apparatus 12 and analyzes the collected health data. The generation unit is implemented, for example, by the specific processing unit 290 of the data processing apparatus 12 and generates advice based on the analysis result. The provision unit is implemented, for example, by a control unit 46A of the robot 414 and provides relaxation or mental health guidance based on the generated advice. The correspondence between each unit and the apparatus or control unit is not limited to the above examples and various modifications are possible.

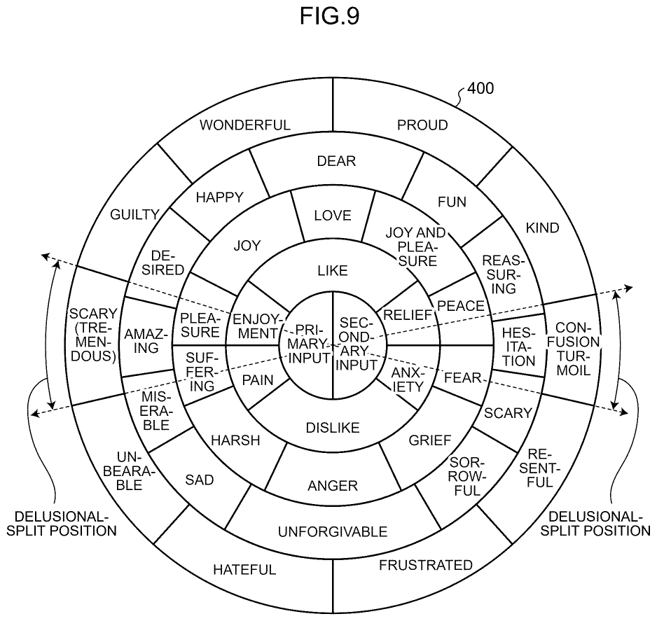

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

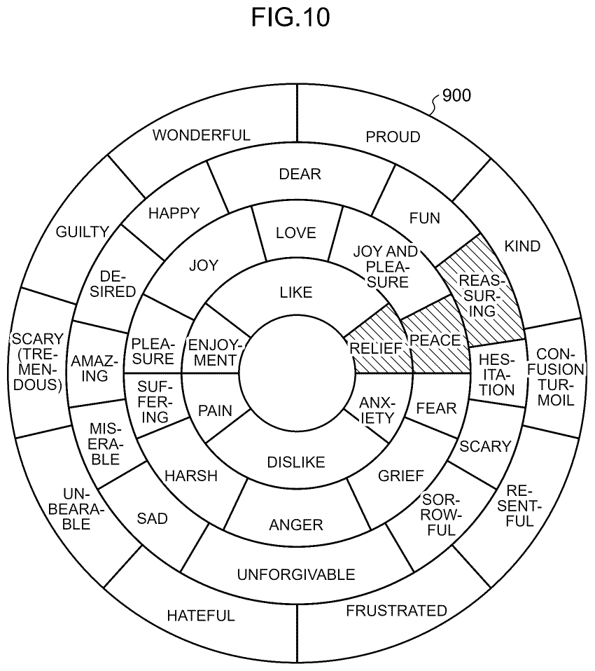

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.
(Supplementary Note 1)A system comprising: a collection unit configured to collect health data; an analysis unit configured to analyze data collected by the collection unit; a generation unit configured to generate advice based on an analysis result obtained by the analysis unit; and a provision unit configured to provide relaxation or mental health guidance based on the advice generated by the generation unit.
(Supplementary Note 2)The system according to Supplementary Note 1, wherein the collection unit comprises a heart rate sensor or a sensor configured to measure a stress level.
(Supplementary Note 3)The system according to Supplementary Note 1, wherein the analysis unit analyzes the collected health data and evaluates a health condition of a user.
(Supplementary Note 4)The system according to Supplementary Note 1, wherein the generation unit generates appropriate exercise or nutrition advice based on the health condition of the user.
(Supplementary Note 5)The system according to Supplementary Note 1, wherein the provision unit provides relaxation or mental health guidance based on a stress level of the user.
(Supplementary Note 6)The system according to Supplementary Note 1, wherein the collection unit estimates an emotion of the user and adjusts a timing for collecting health data based on the estimated emotion of the user.
(Supplementary Note 7)The system according to Supplementary Note 1, wherein the collection unit analyzes past health data of the user and selects an appropriate collection method.
(Supplementary Note 8)The system according to Supplementary Note 1, wherein the collection unit performs filtering based on a current activity status or environment of the user when collecting health data.
(Supplementary Note 9)The system according to Supplementary Note 1, wherein the collection unit estimates an emotion of the user and determines a priority of health data to be collected based on the estimated emotion of the user.
(Supplementary Note 10)The system according to Supplementary Note 1, wherein the collection unit considers a geographic location information of the user when collecting health data and preferentially collects highly relevant data.
(Supplementary Note 11)The system according to Supplementary Note 1, wherein the collection unit analyzes a user's social media activity when collecting health data and collects relevant data.
(Supplementary Note 12)The system according to Supplementary Note 1, wherein the analysis unit estimates an emotion of the user and adjusts a method of expressing analysis based on the estimated emotion of the user.
(Supplementary Note 13)The system according to Supplementary Note 1, wherein the analysis unit adjusts a level of detail of analysis based on an importance of health data during analysis.
(Supplementary Note 14)The system according to Supplementary Note 1, wherein the analysis unit applies different analysis algorithms according to a category of health data during analysis.
(Supplementary Note 15)The system according to Supplementary Note 1, wherein the analysis unit estimates an emotion of the user and adjusts a length of analysis based on the estimated emotion of the user.
(Supplementary Note 16)The system according to Supplementary Note 1, wherein the analysis unit determines a priority of analysis based on a timing of health data collection during analysis.
(Supplementary Note 17)The system according to Supplementary Note 1, wherein the analysis unit adjusts an order of analysis based on a relevance of health data during analysis.
(Supplementary Note 18)The system according to Supplementary Note 1, wherein the generation unit estimates an emotion of the user and adjusts a method of expressing advice based on the estimated emotion of the user.
(Supplementary Note 19)The system according to Supplementary Note 1, wherein the generation unit adjusts a level of detail of advice based on an importance of health condition when generating advice.
(Supplementary Note 20)The system according to Supplementary Note 1, wherein the generation unit applies different advice algorithms according to a category of health condition when generating advice.
(Supplementary Note 21)The system according to Supplementary Note 1, wherein the generation unit estimates an emotion of the user and adjusts a length of advice based on the estimated emotion of the user.
(Supplementary Note 22)The system according to Supplementary Note 1, wherein the generation unit determines a priority of advice based on a change in health condition when generating advice.
(Supplementary Note 23)The system according to Supplementary Note 1, wherein the generation unit adjusts an order of advice based on a relevance of health condition when generating advice.
(Supplementary Note 24)The system according to Supplementary Note 1, wherein the provision unit estimates an emotion of the user and adjusts a method of expressing relaxation or mental health guidance based on the estimated emotion of the user.
(Supplementary Note 25)The system according to Supplementary Note 1, wherein the provision unit analyzes past stress levels of the user when providing guidance and selects optimal guidance.
(Supplementary Note 26)The system according to Supplementary Note 1, wherein the provision unit customizes a means of guidance based on a current living situation of the user when providing guidance.
(Supplementary Note 27)The system according to Supplementary Note 1, wherein the provision unit estimates an emotion of the user and determines a priority of guidance based on the estimated emotion of the user.
(Supplementary Note 28)The system according to Supplementary Note 1, wherein the provision unit considers a geographic location information of the user when providing guidance and selects optimal guidance.
(Supplementary Note 29)The system according to Supplementary Note 1, wherein the provision unit analyzes a user's social media activity when providing guidance and proposes a means of guidance.

What is claimed is:

1. A system comprising:
a communication interface configured to communicate with a client terminal via a packet-switched network;
a memory storing a data generation model obtained by deep learning on a neural network, and an emotion identification model; and
circuitry configured to:
receive, from the client terminal via the communication interface, sensor data comprising at least one of voice data, image data, or time-series biometric data;
estimate an emotion of a user by applying the emotion identification model to the sensor data;
analyze the sensor data using a time-series analysis model comprising at least one of a recurrent neural network or a Transformer-based architecture to extract a pattern feature vector;
detect an anomaly by comparing the pattern feature vector with reference data and generating an anomaly score indicating a degree of deviation;
generate, using the data generation model, inference data based on the anomaly score and the estimated emotion, the inference data comprising at least one of a recommendation label, a probability score, or natural language text; and
transmit the inference data to the client terminal via the communication interface and the packet-switched network, the inference data causing the client terminal to present the inference data to the user.

2. The system according to claim 1, wherein the sensor data comprises health information of the user, the health information comprising at least one of heart rate, blood pressure, stress level, or sleep duration as time-series numerical vectors.

3. The system according to claim 1, wherein the circuitry is further configured to preprocess the sensor data by performing at least one of noise removal, normalization, or feature extraction before analyzing the sensor data.

4. The system according to claim 1, wherein the time-series analysis model comprises a long short-term memory network or a Transformer model with a self-attention mechanism, and wherein the circuitry is configured to extract the pattern feature vector by applying feature extraction layers to multidimensional time-series tensor data.

5. The system according to claim 1, wherein the circuitry is further configured to detect the anomaly by applying at least one of threshold judgment, autoencoder-based anomaly scoring, or rule-based branching to the anomaly score.

6. The system according to claim 1, wherein the circuitry is further configured to adjust a timing of receiving the sensor data from the client terminal based on the estimated emotion, such that when the estimated emotion indicates stress, the circuitry increases a frequency of receiving the sensor data, and when the estimated emotion indicates relaxation, the circuitry maintains a normal frequency.

7. The system according to claim 1, wherein the circuitry is further configured to analyze past sensor data stored in a database associated with the user to select an optimal data collection method using at least one of a decision tree, a random forest, or a reinforcement learning model.

8. The system according to claim 1, wherein the circuitry is further configured to determine a priority of the sensor data to be received based on the estimated emotion, such that when the estimated emotion indicates stress, the circuitry prioritizes sensor data having a high importance attribute.

9. The system according to claim 1, wherein the circuitry is further configured to receive geographic location information from the client terminal and to preferentially receive sensor data associated with a geographic region corresponding to the geographic location information.

10. The system according to claim 1, wherein the circuitry is further configured to adjust an expression style of the inference data based on the estimated emotion, such that when the estimated emotion indicates stress, the inference data is generated in a simplified expression style, and when the estimated emotion indicates relaxation, the inference data is generated in a detailed expression style.

11. The system according to claim 1, wherein the circuitry is further configured to calculate an importance score for each item of the sensor data and to adjust a level of detail of analysis based on the importance score, such that detailed analysis is performed for sensor data having a high importance score and simplified analysis is performed for sensor data having a low importance score.

12. The system according to claim 1, wherein the circuitry is further configured to apply different analysis algorithms according to a category of the sensor data.

13. The system according to claim 1, wherein the circuitry is further configured to adjust a length of the inference data based on the estimated emotion, such that when the estimated emotion indicates stress, the circuitry generates concise inference data, and when the estimated emotion indicates relaxation, the circuitry generates detailed inference data.

14. The system according to claim 1, wherein the circuitry is further configured to determine a priority of generating the inference data based on a submission timing associated with the sensor data, such that sensor data having a more recent submission timing is analyzed with a higher priority.

15. The system according to claim 1, wherein the inference data comprises guidance data for at least one of relaxation or mental health, and wherein the circuitry is further configured to adjust content of the guidance data based on the estimated emotion.

16. The system according to claim 1, wherein the circuitry is further configured to analyze social media activity data of the user received from the client terminal, and to adjust the inference data based on the analyzed social media activity data.

17. The system according to claim 1, wherein the circuitry is further configured to determine a priority of the inference data based on the estimated emotion, such that when the estimated emotion indicates stress, the circuitry prioritizes inference data associated with a high urgency attribute.

18. A system comprising:
a communication interface configured to communicate, via a packet-switched network conforming to at least one of a 5G, Wi-Fi, or Bluetooth communication standard, with a client terminal comprising a touch panel, a microphone, a speaker, a camera having a CMOS image sensor, and a display;
a processor;
a random-access memory;
a memory storing a data generation model obtained by deep learning on a neural network, and an emotion identification model; and
circuitry configured to:
receive, from the client terminal via the communication interface, sensor data comprising at least one of voice data captured by the microphone, image data captured by the camera, or time-series biometric data from a wearable device communicatively coupled to the client terminal;
preprocess the sensor data by performing at least one of noise removal, normalization, or feature extraction;
estimate an emotion of the user by applying the emotion identification model to the sensor data;
analyze the preprocessed sensor data using a time-series analysis model comprising at least one of a recurrent neural network, a long short-term memory network, or a Transformer model to extract a pattern feature vector;
detect an anomaly by comparing the pattern feature vector with reference data and generating an anomaly score;
generate, using the data generation model, inference data based on the anomaly score and the estimated emotion, the inference data comprising at least one of a recommendation label, a probability score, or natural language text;
adjust at least one of a level of detail, an expression style, or a length of the inference data based on the estimated emotion; and
transmit the inference data to the client terminal via the communication interface, the inference data causing the client terminal to present the inference data to the user via at least one of the display or the speaker.

19. The system according to claim 18, wherein the data generation model comprises at least one of a text generation AI, an image generation AI, or a multimodal generation AI, and wherein the data generation model is a fine-tuned model configured to output inference results from prompts without instructions.

20. A method performed by circuitry of a system comprising a communication interface, a memory storing a data generation model obtained by deep learning on a neural network and an emotion identification model, the method comprising:
receiving, from a client terminal via the communication interface and a packet-switched network, sensor data comprising at least one of voice data, image data, or time-series biometric data;
estimating an emotion of a user by applying the emotion identification model to the sensor data;
analyzing the sensor data using a time-series analysis model comprising at least one of a recurrent neural network or a Transformer-based architecture to extract a pattern feature vector;
detecting an anomaly by comparing the pattern feature vector with reference data and generating an anomaly score indicating a degree of deviation;
generating, using the data generation model, inference data based on the anomaly score and the estimated emotion, the inference data comprising at least one of a recommendation label, a probability score, or natural language text; and
transmitting the inference data to the client terminal via the communication interface and the packet-switched network, the inference data causing the client terminal to present the inference data to the user.
